(19) United States
(12) Patent Application Publication
(10) Pub. No.: US 2019/0216574 A1
(43) Pub. Date: Jul. 18, 2019

(54) System for Marking the Surface of a Patient's Tissue in the Course of Imaging

(71) Applicants: Guy's and St. Thomas' NHS Foundation Trust, London (GB); NPL Management Limited, Teddington (GB)

(72) Inventors: Andrew John Coleman, London (GB); Edward Benjamin Hughes, Teddington (GB); Timothy James Coveney, West Molesey (GB); Richard Anthony Edward Page, Ketton (GB); Richard Smith, Gamlingay (GB); Neil Anthony Litten, Reading (GB); Mayur Arunkumar Patel, Watford (GB); Paul Antony Merritt, Royston (GB)

(21) Appl. No.: 16/362,099

(22) Filed: Mar. 22, 2019

Related U.S. Application Data
(63) Continuation of application No. PCT/GB2017/052848, filed on Sep. 22, 2017.

(30) Foreign Application Priority Data
Sep. 22, 2016 (GB) 1616153.1

(51) Int. Cl. A61B 90/00; A61B 5/00

(52) U.S. Cl. CPC A61B 90/39; A61B 5/0077; A61B 5/742; A61B 2090/3983; A61B 2090/3908; A61B 2090/395; A61B 5/444

(57) ABSTRACT

Marking apparatus for marking tissue imaged through an imaging device including a viewing element in the form of a spacer having an imaging window which provides an imaging zone through which tissue can be imaged by an imaging probe. The apparatus can include a marker implement connected to a coupling device. In some embodiments, the coupling device is configurable to position the marker implement in a first position substantially out of the imaging window and a second position in the imaging zone of the imaging window. The marker implement can apply a mark when in the second position and thereby to tissue in the imaging zone while the apparatus remains in the imaging position on the tissue. The spacer can be length adjustable in order to change the depth of focus of the imaging probe, thereby to focus at different depths within a patient's skin.

### CROSS-REFERENCE TO RELATED APPLICATIONS

This application is a continuation of International Application No. PCT/GB2017/052848 having an international filing date of Sep. 22, 2017 entitled “System for Marking the Surface of a Patient's Tissue in the Course of Imaging”. The '848 international application claimed priority benefits, in turn, from United Kingdom Patent Application No. 1616153.1 filed on Sep. 22, 2016.

The '848 international application and the '153.1 UK application are hereby incorporated by reference herein in their entireties.

### FIELD OF THE INVENTION

The present invention relates to a marking apparatus for marking the location of tissue imaged through an imaging device, as well as to a system for imaging and marking tissue. The marking apparatus is particularly useful for marking the location or delineating the margin of a clinical feature of interest present on or under the surface of a patient's skin. Another aspect of the invention also provides an adjustable spacer for such a system.

Basal cell carcinoma (BCC) accounts for around 75% of all skin cancers. A recent study shows that BCC is the most common malignant disease in the UK, with the number of cases having risen by 80% in a decade. Most BCCs and other skin cancers are treated by surgical removal of the cancerous region.

The current method for surgical removal of malignant or cancerous regions involves identifying a ‘cancer margin’, the region within which the skin cancer (basal or squamous cell carcinomas) is present. The ‘surgical margin’, the region to be removed, is subsequently established. The surgeon marks the ‘surgical margin’ directly on the surface of the skin using a handheld felt tip marker pen. These pens are normally sterile and individually packaged and use a surgical grade ink, typically some formulation of gentian violet. The sterilised felt tip marker pen is used to mark the surface of the skin so as to delineate the identified ‘surgical margin’. Desired skin flaps, needed to repair the wound, are often also marked using the marker pen.

Marking is typically guided by visual clinical examination. The ‘surgical margin’, marked for surgical removal of the skin cancer, requires the surgeon to judge the ‘cancer margin’ by eye. The ‘surgical margin’ includes the cancer present on the surface of the skin visible to the eye with an added safety margin of skin (often between 3-10 mm) around the visible ‘cancer margin’. The safety margin is selected to ensure surgical removal of any invisible infiltration of the disease. Sometimes, however, the disease will have infiltrated to underlying layers of tissue, beyond the safety margin.

A problem with surgical removal of skin cancers is determining the amount of tissue to cut out around the cancer. Using a hand-held marker pen and marking the ‘surgical margin’ by eye has several drawbacks. The lack of imaging means that if the cancer has extended laterally under the surface of the skin, these extensions will not be seen. As a consequence, the ‘surgical margin’ will not contain all of the cancer and surgical removal will be incomplete, causing the cancer to return. This often requires a costly secondary operation, further treatment and delay of effective treatment. If too much is cut out, the treatment is unnecessarily aggressive, leading to complications which may require continuing expensive treatment. Furthermore, healthy tissue is removed, leaving a larger wound than necessary that may look unsightly.

Non-invasive imaging technologies can assist the naked eye in identifying the size of the cancerous tissue by showing to the surgeon the otherwise invisible lateral extensions of the cancer underneath the surface of the skin. Imaging technologies can also help by providing a better view of the lesion on the skin surface by magnification and the reduction of surface glare (as in dermoscopy). Examples of non-invasive imaging technologies currently used in assisting the naked eye in defining the cancer margin include optical coherence tomography (OCT), dermoscopy, reflectance confocal microscopy and ultrasound imaging.

However, imaging technologies are not used routinely as an aid in surgical margin marking due to the difficulty in marking the skin or tissue in practice. In order to image tissue sections of interest, the imaging probe must be in contact with the surface of the skin. Once the imaging probe is lifted off the skin or moved to the side, which is necessary to leave room to make a mark with a pen, the image is lost and the surgeon must estimate from memory the precise location of the tissue that was imaged. The advantages of new optical imagining technologies in accurately tracking the lateral extension of cancers are lost.

Examples of prior art skin markers used with image guidance can be found in US-2007/0225605, US-2014/0005542, US-2006/0106312 and U.S. Pat. No. 6,745,067.

### SUMMARY OF INVENTION

The present disclosure provides an improved marking apparatus for marking the location of imaged tissue sections, an improved system for imaging and marking, as well as a configurable spacer for such a system.

According to an aspect of the present disclosure, there is provided marking apparatus for marking tissue imaged through an imaging device, including: a viewing element including an imaging window providing an imaging zone through which tissue can be imaged; a marker implement; a coupling device to which the marker implement is connected; the coupling device being configurable to position the marker implement in a first position substantially out of the imaging window and a second position in the imaging zone of the imaging window; the marker implement being able to apply a mark when in the second position and thereby to mark the tissue in the imaging zone while the apparatus remains in position on the tissue.

The apparatus and system disclosed herein provides a mechanism by which it is possible to apply a mark on the surface of imaged tissue (such as skin) while keeping the imaging device in position on the tissue, thereby enabling the mark to be applied under image-guidance. This is not possible with the prior art systems referred to above. The physician can apply a mark accurately, without the need to have to estimate from memory the precise location of the tissue being imaged.

Advantageously, the apparatus includes a guide member to which the marker implement is coupled, the guide member being structured to move the marker implement in a direction substantially orthogonal to the imaging window.

By enabling the marker implement to approach the tissue to be marked at a right angle to the tissue can ensure the accurate placement of a mark at the location of the tissue being imaged, in other words precisely in the position which the physician identifies on screen. It is not excluded, however, that the marker implement may be moved in a non-orthogonal direction, while in other embodiments it may not move at all from the second position, for instance in cases where the implement is able to spray or otherwise apply an ink mark from a distance. In some embodiments, the marker implement may be arranged to move in a diametrical direction, for instance at an acute angle to the skin. This can be achieved by appropriate design of the moving mechanism.

In some preferred embodiments, the guide member is operable to move the marker implement so as to extend through the imaging window to apply a mark to tissue. In other words, the marker implement can be extended beyond the window to press upon tissue held against the imaging window.

In some embodiments, the guide member includes a first guide channel section substantially orthogonal to the imaging window. The guide member can include a second guide channel section substantially parallel to the imaging window, the second guide channel section providing for movement of the marker implement in a direction substantially parallel to the imaging window from the first to the second positions.

In some preferred embodiments, the marker implement is removable, from the marking apparatus, for example so as to enable the marker implement to be changed or to be disposed.

Providing a marking apparatus where the marker implement is removable enables easy replacement of the marker implement to prevent cross-contamination between uses.

In some preferred embodiments, the marking apparatus is integral with an imaging device, particularly built-in to the casing of the imaging device. At least the viewing element and imaging window are preferably integral with a casing of the imaging device.

In some embodiments, the marking apparatus can be removable from the imaging device, enabling periodic maintenance and cleaning of the whole system and of the imaging device.

In some embodiments, the marking apparatus can include a support structure attachable to a casing of an imaging device.

The support structure can be used in some embodiments to support the pin guides which assist in positioning the marking apparatus on tissue. The support structure can also act as a housing, to protect the components of the apparatus.

In some preferred embodiments, the viewing element is in the form of a spacer element, the spacer element including a front face which incorporates the imaging window. Advantageously, the spacer element can include front and rear faces, and can include a length adjusting mechanism which can adjust a distance between the front and rear faces; wherein a change in said distance effects a change in focal position of the imaging device.

The ability to vary focal length can enable adjustment for different imaging devices but more importantly the ability to focus into the depth of skin tissue so as to locate diseased tissue underneath the skin surface.

In some preferred embodiments, the length adjusting mechanism can adjust the distance between the front and rear faces from 0 mm to substantially 20 mm.

In some preferred embodiments, the length adjusting mechanism can adjust the distance between the front and rear faces from substantially 5 mm to substantially 11 mm.

In some preferred embodiments, the spacer element is integral with the marking apparatus. This advantageously provides a ready fitted device requiring no set-up prior to use of the device.

The apparatus can advantageously include a triggering or actuating mechanism (herein after referred to as triggering mechanism) connected to the coupling device for moving the marker implement between the first and second positions. In some preferred embodiments, the triggering mechanism is operable to move the marker implement to extend through the imaging window.

According to another aspect of the disclosure, there is provided a system for imaging tissue and marking imaged tissue, including: an imaging device; marking apparatus coupled to the imaging device; and a display system coupled to the imaging device and through which images obtained from the imaging device can be displayed; wherein the marking apparatus includes: a viewing element including an imaging window providing an imaging zone through which tissue can be imaged; a marker implement; a coupling device to which the marker implement is connected; the coupling device being configurable to position the marker implement in a first position substantially out of the imaging window and a second position in the imaging zone of the imaging window; the marker implement being able to apply a mark when in the second position and thereby to tissue in the imaging zone while the apparatus remains in position on the tissue.

In at least some embodiments with the system, as a physician moves the imaging device over different tissue sections, an image of the tissue section is displayed on the display system. As a feature of interest appears on the display system, the physician can activate the triggering mechanism, causing the marking apparatus to place a mark on the tissue section being imaged.

Advantageously, the system can include a feedback device operable to generate a feedback signal once a mark has been applied by the marking implement.

In at least some embodiments, the imaging device is most suitable for use with an OCT imaging device.

According to another aspect of the present disclosure, there is provided a configurable viewing element for an imaging device, the viewing element can include a spacer element provided with a front face and a rear face, an imaging window being disposed in the front face and providing an imaging zone through which tissue can be imaged, and a length adjustment mechanism coupled to the spacer element and operable to adjust a distance between the front and rear faces so as in use effects a change in focal position of an imaging device.

In some preferred embodiments, the length adjustment mechanism is operable to adjust the distance between the front and rear faces from substantially 5 mm to substantially 11 mm.

In some embodiments, he viewing element advantageously includes a housing member having a viewing passage passing therethrough from the rear to the front faces.

In some preferred embodiments, the imaging window is an opening in the viewing element.

The apparatus and system disclosed herein can be used to mark the location of imaged tissue sections. A mark is applied without needing to lift off or move the marking apparatus to the side in order to make room to apply the mark. The mark can therefore accurately indicate the location of any identified features of interest. The marking apparatus described herein can be used to delineate a cancer margin, including any cancer extensions infiltrated to underlying tissue sections, not visible to the naked eye. An appropriate surgical margin can then be marked for removal.

Other features and advantages of the system and apparatus disclosed herein will become apparent from the specific description which follows.

### BRIEF DESCRIPTION OF THE DRAWINGS

Embodiments of the present invention are described below, by way of example only, with reference to the accompanying drawings.

FIG. 1 is a view in side elevation of an embodiment of marking apparatus.

FIG. 2 is a view in front elevation of a marking apparatus.

FIG. 3 is a cross sectional detailed view of a marking apparatus with a marker implement in a first position.

FIG. 4 is a cross-sectional detailed view of a marking apparatus with a marker implement in an extended position.

FIG. 5 is a front perspective view of a marking apparatus with a marker implement in a first position.

FIG. 6 is a cross-sectional view of a marking apparatus.

FIG. 7 is a detailed view in front elevation of a marking apparatus with a marker implement in a first position.

FIG. 8 is a front perspective view of a marking apparatus with a marker implement in an extended position.

FIG. 9 is a cross-sectional view of a marking apparatus.

FIG. 10 is a detailed view in front elevation of a marking apparatus with a marker implement in an extended position.

FIG. 11 is a view in side elevation of a marking apparatus with a marker implement in a first position.

FIG. 12 is a view in side elevation of a marking apparatus with a marker implement in an extended position.

FIG. 13 is a cross-sectional view in side elevation of a marking apparatus with a marker implement in a first position.

FIG. 14 is a cross-sectional view in side elevation of a marking apparatus with a marker implement in an extended position.

FIGS. 15A-15D are views of a marking apparatus with a marker implement in an extended position.

FIG. 16 is a cross-sectional front perspective view of another embodiment of a marking apparatus.

FIG. 17 is a view in side elevation of another embodiment of a marking apparatus.

FIG. 18 is a view in side elevation of another embodiment of a marking apparatus.

FIG. 19 is a view in side elevation of the marking apparatus of FIG. 17.

FIG. 20 is a view in side elevation of the marking apparatus of FIG. 18.

FIG. 21 is a cross-sectional view in side elevation of the marking apparatus of FIG. 18.

FIG. 22 is a cross-sectional view in side elevation of the marking apparatus of FIG. 18.

FIG. 23 is a front perspective view of the marking apparatus of FIG. 17.

FIG. 24 is a front perspective view of the marking apparatus of FIG. 18.

FIG. 25 is a top elevation view of the marking apparatus of FIG. 17.

FIG. 26 is a bottom elevation view of the marking apparatus of FIG. 17.

FIG. 27 is a top elevation view of the marking apparatus of FIG. 18.

FIG. 28 is a bottom elevation view of the marking apparatus of FIG. 18.

FIG. 29 is a cross-sectional detailed view of another embodiment of a marking apparatus.

FIG. 30 is a view in side elevation of another embodiment of marking apparatus.

FIG. 31 is a view in front elevation of a marking apparatus.

FIG. 32 is a cross-sectional detailed view of a marking apparatus with a marker implement in a first position.

FIG. 33 is a cross-sectional detailed view of a marking apparatus with a marker implement in an extended position.

FIG. 34 is a front perspective view of marking apparatus with a marker implement in a first position.

FIG. 35 is a view in side elevation of marking apparatus with a marker implement in a first position.

FIG. 36 is a front perspective view of a marking apparatus with a marker implement in an extended position.

FIG. 37 is a view in side elevation of a marking apparatus.

FIG. 38 is a detailed view in front elevation of a marking apparatus with a marker implement in an extended position.

FIG. 39 is a view in side elevation of a marking apparatus with a marker implement in a first position.

FIG. 40 is a view in side elevation of a marking apparatus with a marker implement in an extended position.

FIGS. 41A-41D are views of a marking apparatus with a marker implement in a first position.

FIGS. 42A-42D are views of a marking apparatus with a marker implement in an extended position.

FIG. 43 is a cross-sectional front perspective view of a marking apparatus.

FIG. 44 is a view in side elevation of a marking apparatus.

### DETAILED DESCRIPTION OF ILLUSTRATED EMBODIMENT(S)

Described below are various embodiments of a marking apparatus for marking the location of imaged tissue. The embodiments described below are examples only, the specific form of the components described herein not being essential to the teachings herein. The imaging device is not described in detail herein as the teachings herein are applicable to known imaging devices.

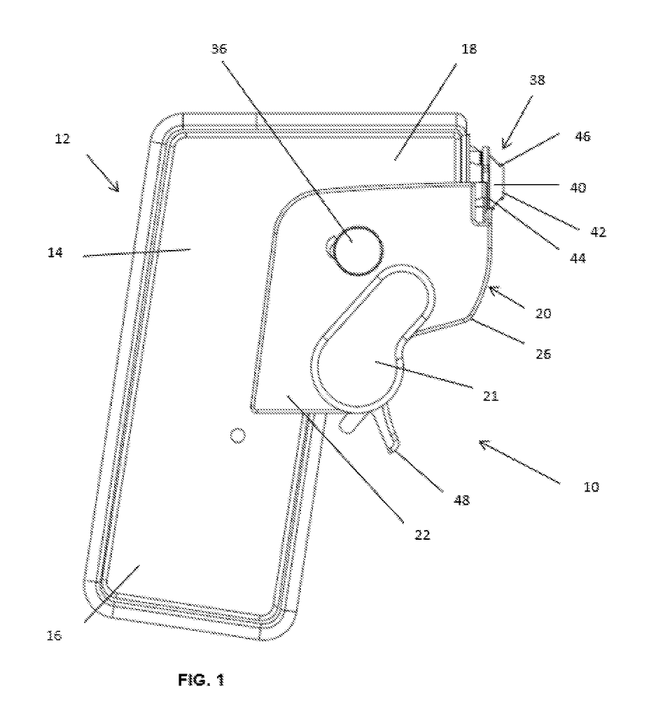

The embodiments described below and shown in FIGS. 1 to 29 are particularly suited for use with an OCT imaging device.

Referring first to FIG. 1, this shows an example of marking apparatus 10 coupled to imaging device 12 for imaging tissue. The imaging, or probe, device 12 can be of known type and in this example is a hand-held unit shaped so as to be able to be gripped by a user with one hand. Imaging device 12 includes probe housing 14 having hand grip portion 16 and head 18 at which an imaging probe head of known type is located. The probe head is not visible in FIG. 1.

The embodiment of marking apparatus 10 shown is designed to fit and attach to imaging device 12 and specifically at head end 18 of probe housing 14. The marking apparatus includes support structure 20 with two side panels 22 and 24 which lie alongside respective sides of probe housing 14. The two side panels 22 and 24 are connected together by front panel 26. The two side panels 22 and 24 are in this embodiment made of plastics material and preferably as a single piece. They are preferably smooth and flat or gently curved, so that they can easily be wiped clean, as well as being shaped to fit the contour of probe housing 14, leaving sufficient space for housing and preferably supporting the internal components of the marking assembly.

Two additional housing members 21 and 23, which in this embodiment are in the form of flat panels, are attached to respective side panels 22 and 24.

A drive mechanism or moving mechanism 36 is also provided at side panel 22. As illustrated in this embodiment, moving mechanism 36 can be, for example a toothed wheel fitting within a partially toothed slot (not visible in FIG. 1) and a turning knob. In some embodiments, a similar thumbwheel and toothed slot is preferably provided on the other side of apparatus 10 at side panel 24. As is described below, thumbwheels 36 can be used to move the apparatus away from and towards the head of the imaging probe for focusing purposes.

In the embodiment illustrated in FIG. 1, apparatus 10 also includes spacer 38, located on the front of marking apparatus 10 and in practice is disposed in front of the probe head of imaging device 12. In some embodiments, spacer 38 includes housing 40 having front face 42 and rear face 44. Housing 40 has, in this example, a through aperture such that the probe head of device 12 retains imaging access through marking apparatus 10. The through aperture is substantially circular, although could have different shapes. In this example, housing 40 is telescopically extendable and retractable. Front face 42 is in this embodiment substantially flat for placement against tissue, although in other embodiments can be gently curved. Housing 40 has in the embodiment shown a tapering front section 46 for ergonomic purposes. It will be appreciated, that housing 40 can have other suitable shapes.

Spacer 38 can be of plastics material or another suitable material that can be cleaned or sterilised.

Marking apparatus 10 can also include triggering mechanism 48, which in this embodiment is a trigger button, positioned at the front of apparatus 10 and is of a type which can be actuated by a user's finger.

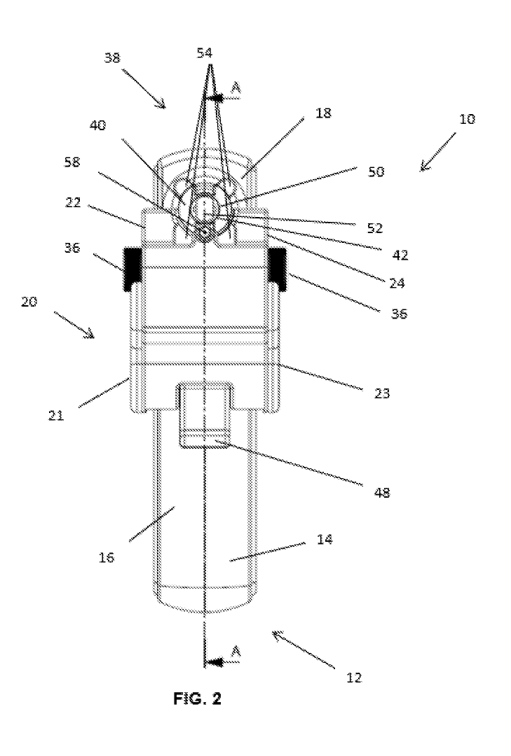

FIG. 2 is a view in front elevation of the assembly shown in FIG. 1. Spacer 38 can be seen located at the front of marking apparatus 10 and includes window 50 in font face 42. Widow 50 is aligned with the probe head such that the probe head has imaging access through spacer 38 and window 50. Window 50 thus provides imaging zone 52 within its perimeter. In some embodiments, spacer 38 is attached to casing 14 of imaging device 12 using four fixing screws 54. Fixing screws 54 can provide running pillars to maintain alignment of spacer 38 during movement.

Marking apparatus 10 can also include marker implement 58, which can be a type of marker suitable for applying a mark to tissue. In some preferred embodiments, implement 58 is a felt tip marker pen with a nib. In the view of FIG. 2, marker implement 58 is located outside imaging zone 52.

Two thumbwheels 36 can be seen located on opposing sides of apparatus 10, by respective side panels 22 and 24. Two thumbwheels makes the apparatus suitable for left or right handed use. Two housings members 21 and 23 can also be seen located on opposing sides of apparatus 10, by respective side panels 22 and 24.

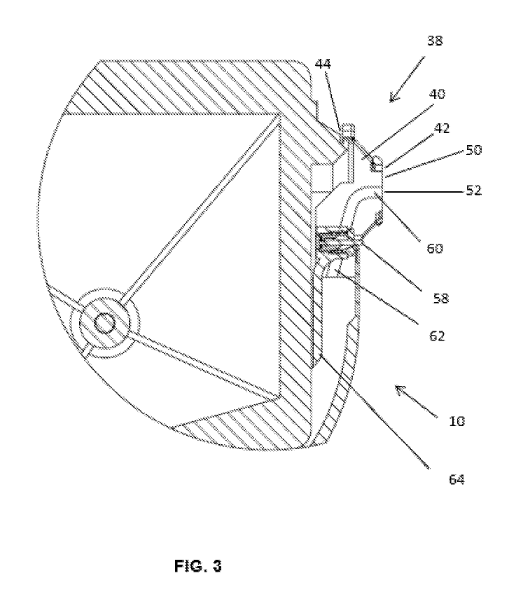

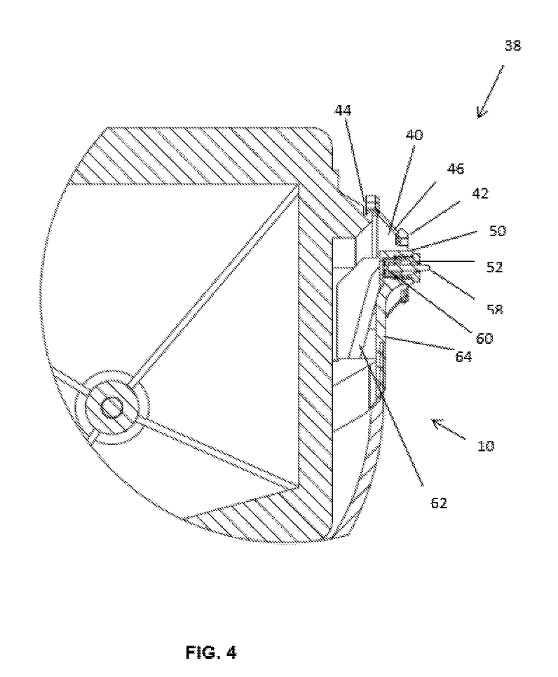

FIGS. 3 and 4 show cross-sectional views of marking apparatus 10 with marker implement 58 in two different operating positions. FIG. 3 shows marker implement 58 in a first position, outside imaging window 50 and imaging zone 52, whereas FIG. 4 shows marker implement 58 extending through imaging window 50 and in a position in which it can apply a mark to skin abutting the front face 42 of spacer 38.

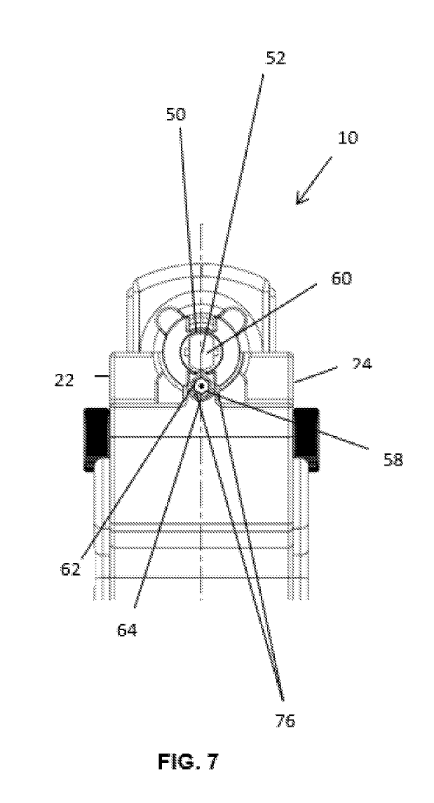

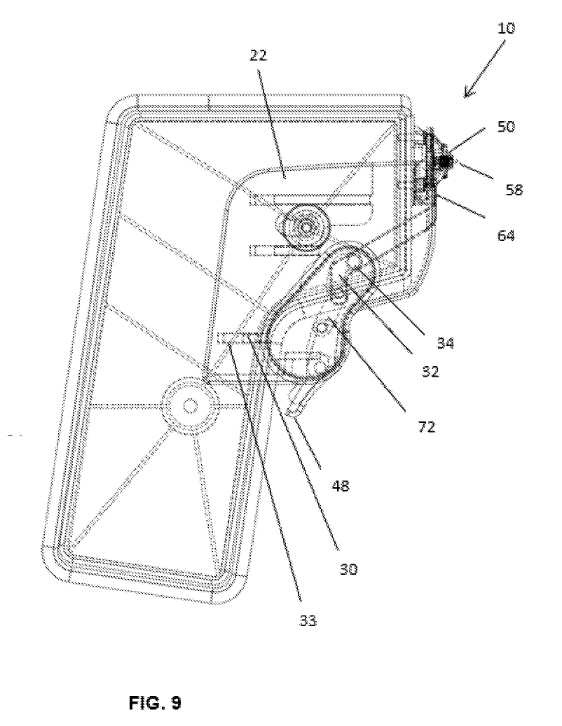

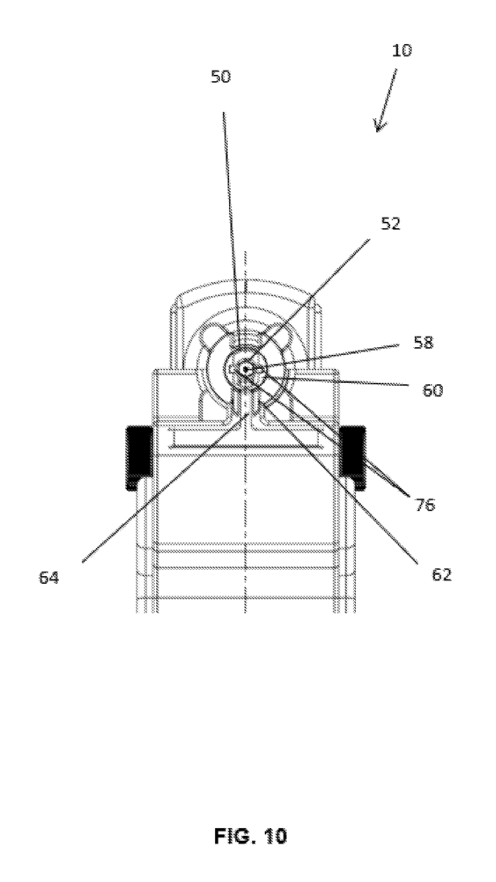

Referring to FIG. 3, apparatus 10 includes a guide member which is provided in this embodiment with a first guide channel section 60 substantially orthogonal to the front face 42 of spacer 38 and a second guide channel section 62 substantially parallel to the front face 42 of spacer 38 or, as illustrated in this embodiment, at an angle to the front face 42 of spacer 38. The first guide channel section 60 and second guide channel section 62 are coupled by a bend section. In practice, the guide member preferably includes opposing channels, as will be apparent, for example, in FIGS. 7 and 10 in particular.

Marker implement 58 is fitted to coupling device 64 which can include guide followers, typically follower pins, which are located in the guide channels and are able to slide therein. In FIG. 3, marker implement 58 is shown in the first position, located outside imaging window 50 and retracted from imaging zone 52. In FIG. 4, the coupling device has been moved along second channel section 62 of the guide member to the bend and then along first channel section 60, such that marker implement 58 is positioned within imaging zone 52 of imaging window 50 and so as to extend beyond imaging window 50. Marker implement 58 is preferably centred on imaging zone 52, although in other embodiments it can be positioned off-centre by a different disposition of the guide member.

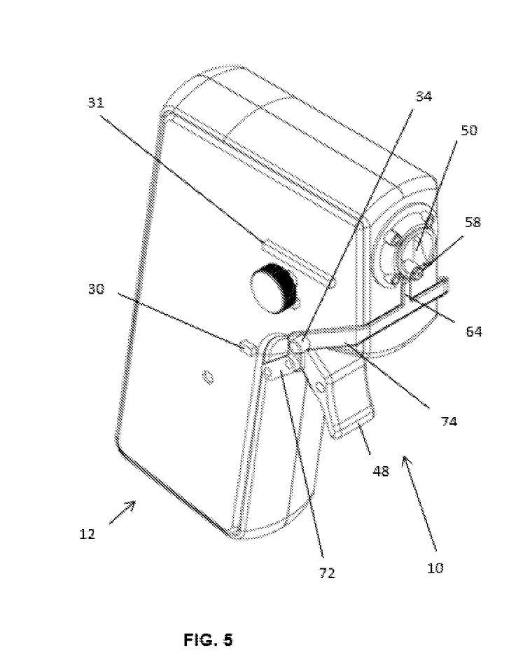

FIGS. 5 to 7 show different views of marking apparatus 10. FIG. 5 shows a front perspective view with side panels 22 and 24 and housing members 21 and 23 removed, to show the internal components of apparatus 10. In FIGS. 5 to 7, marker implement 58 is located in the first position, outside imaging window 50 and in a non-use position. Triggering mechanism 48 can be seen, coupled to drive link 72, which in turn is coupled to drive arm 74 which connects to coupling device 64. Guide features 30 and 31 can also be seen and, in this embodiment, are in the form of rectangular inserts. Drive arm 74 also includes guide pin 34.

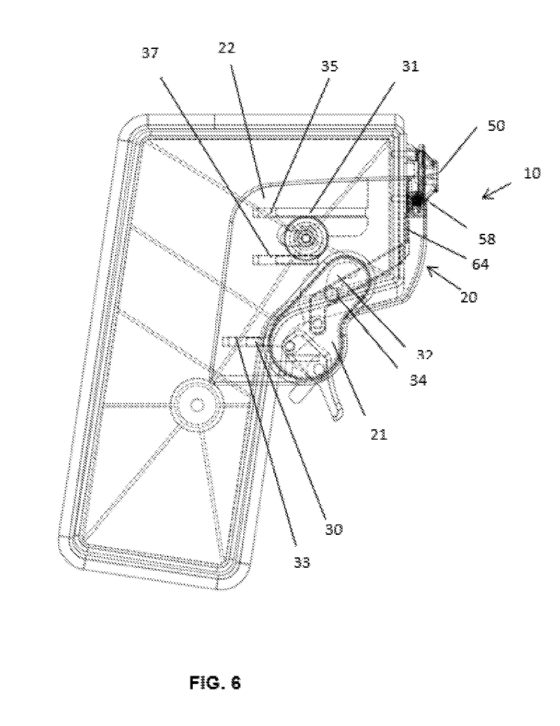

FIG. 6 is a view in side elevation of apparatus 10. Support structure 20 can be seen with side panels 22 and 24 and housing members 21 and 23 in ghost form so as to show the internal components of apparatus 10. Guide feature 32 in side panel 22 is in the form of a slotted aperture or cut-out which curves through ninety degrees and receives guide pin 34. In some preferred embodiments, the opposing side panel 24 has similar guide features (also in the form of apertures or cut-outs) for similar, opposing, guide pins. Guide feature 32 and guide pin 34 help to guide drive arm 74 and in turn coupling device 64 to position correctly marking apparatus 10 on tissue to be marked.

Side panel 22 includes guide features 33 and 35 which in this embodiment are in the form of rectangular slots. Inserts 30 and 31 slide through slots 33 and 35, so as to position support structure 20 in a retracted position and an extended position, as will be described in further detail below. Some embodiments may include additional guide features 37. The marker implement 58 can be seen in the first position and outside imaging window 50.

FIG. 7, shows a front elevation view of marking apparatus 10. Two pins 76 can be seen either side of coupling device 64, which slide in guide channels 60 and 62.

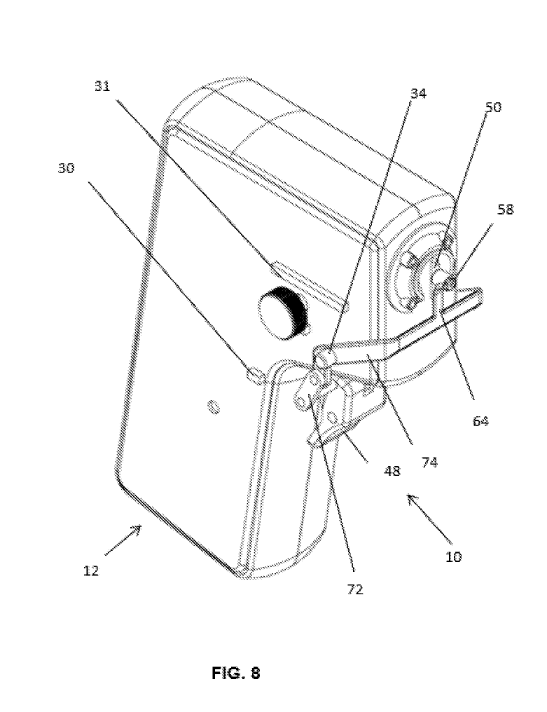

FIGS. 8 to 10 show apparatus 10 with the features of FIGS. 5 to 7 but in which triggering mechanism 48 has been pressed towards the body of probe housing 14. This action causes link element 72 to move upwardly and front-wise, in the view of the Figures, by movement of guide pins 34 along guide feature 32. As a consequence, coupling device 64 to which marker implement 58 is attached moves along guide channels sections 60 and 62, which are in practice shaped similar to the slotted aperture of guide feature 32 in side panels 22 and 24. As will be apparent in FIG. 10, coupling device 64 can be seen sitting in guide channel section 62 with marker implement 58 within imaging window 50. In this embodiment, marker implement 58 is positioned in the centre of imaging zone 52 provided by window 50 so as to extend through and beyond window 50.

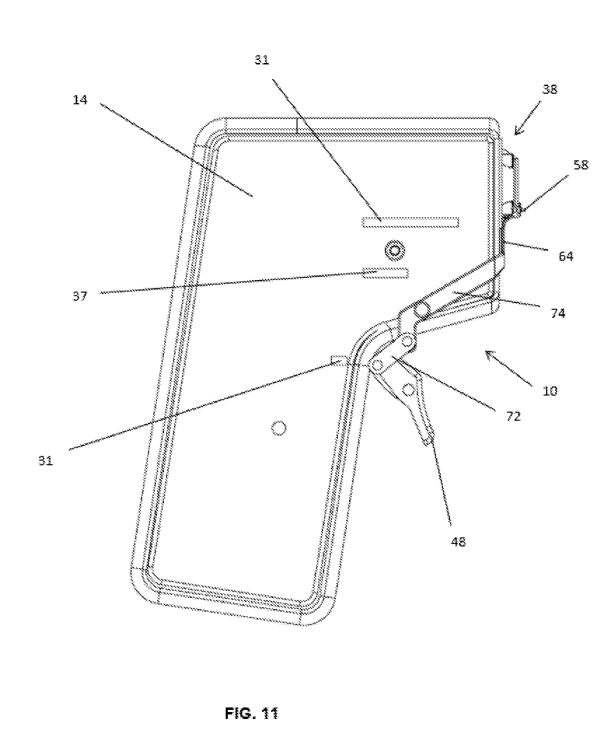

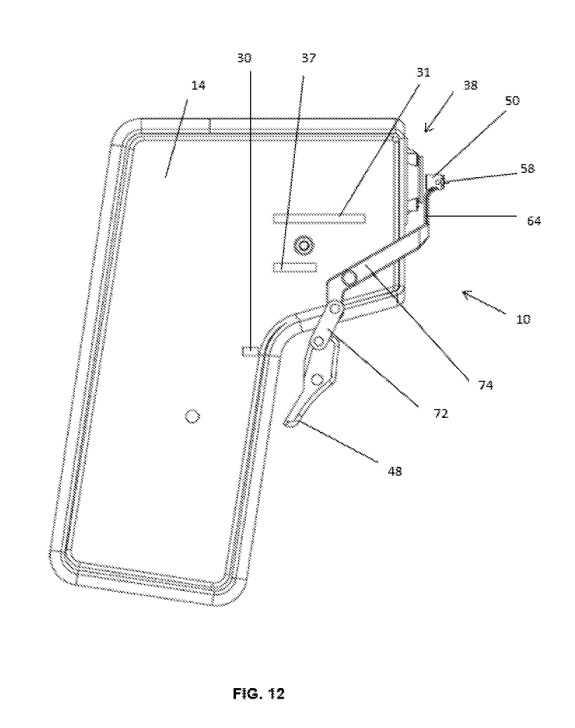

FIGS. 11 and 12 show views of probe housing 14 and of triggering mechanism 48 and coupling device 64 mechanism of marking apparatus 10. In the embodiments show in FIGS. 11 and 12, a portion of spacer 38 has been removed so as to show the two positions of marker implement 58. In FIG. 11 marker implement 58 is in the first position and with triggering mechanism 48 in an inactive position. As can be seen, triggering mechanism 48 is coupled to drive link 72, which in turn is coupled to drive arm 74 and coupling device 64. In FIG. 12 triggering mechanism 48 is shown in an actuated or pressed position, in which it positions drive link 72, drive arm 74 and coupling device 64 in the active configuration, that is so as to extend marker implement 58 beyond imaging window 50. In some preferred embodiments, drive arm 74 and coupling device 64 are in one piece.

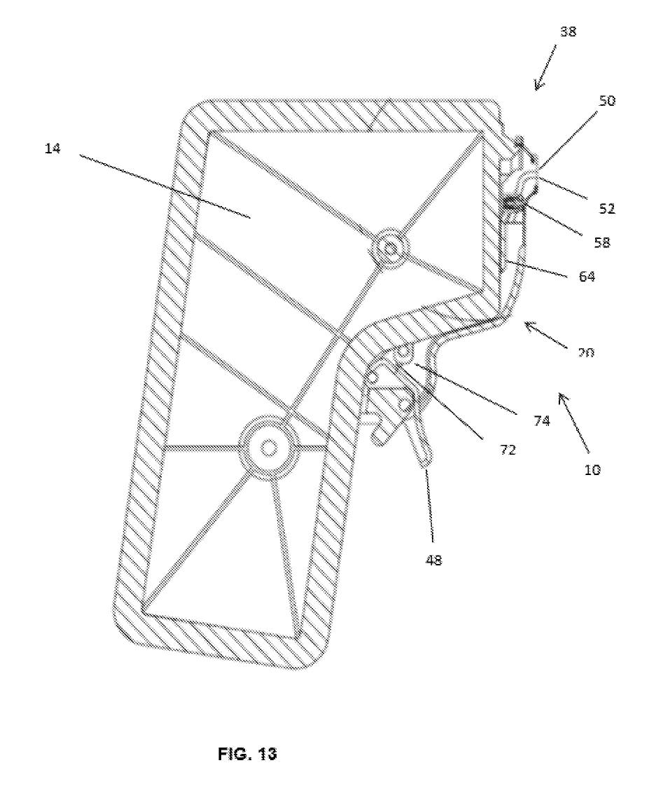

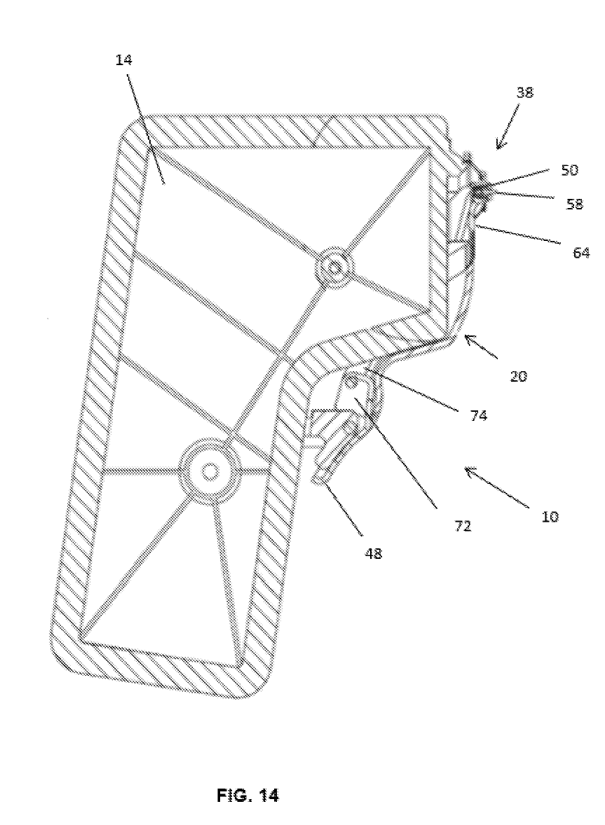

FIGS. 13 and 14 show cross sectional views of probe housing 14 where FIG. 13 shows marker implement 58 in the first position and FIG. 14 shows marker implement 58 in the extended position.

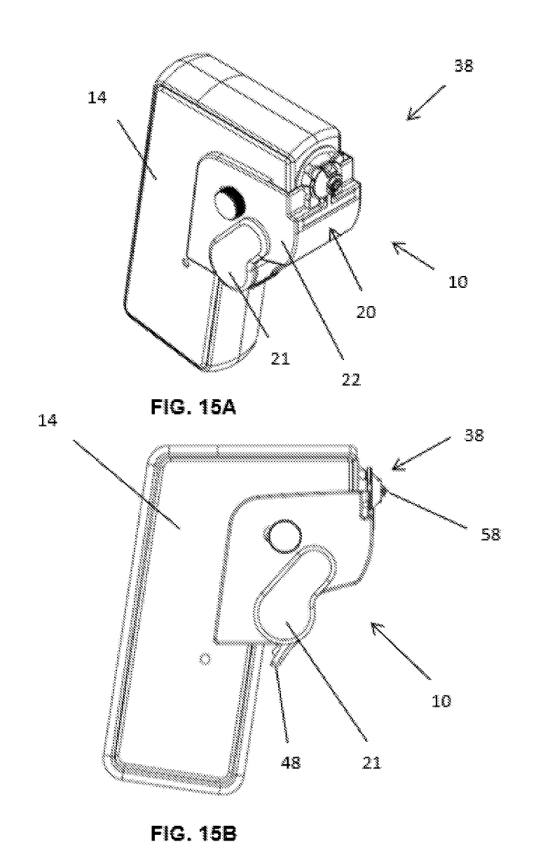

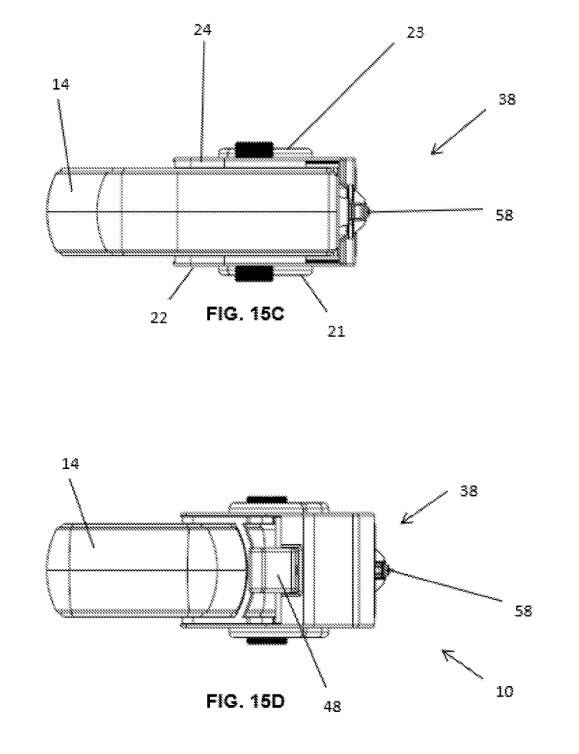

FIGS. 15A-15D show further views of marking apparatus 10 with marker implement 58 in the extended position. FIG. 15A shows marking apparatus 10 in a front perspective view including housing member 21 attached to side panel 22. FIG. 15B shows a side elevation view of marking apparatus 10. FIG. 15C shows marking apparatus 10 from a top perspective view, while FIG. 15D shows marking apparatus 10 from a bottom perspective view. As can be seen most clearly in FIGS. 15b and 15d, triggering mechanism 48 is pressed towards the body of probe housing 14.

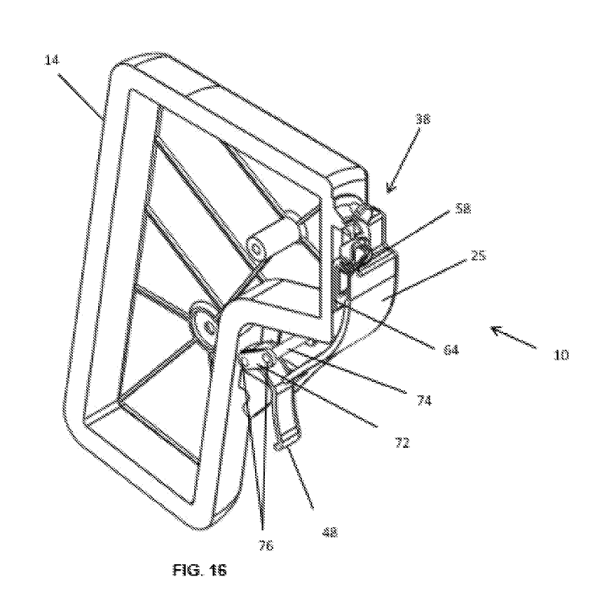

FIG. 16 is a front perspective view of another embodiment of marking apparatus 10 with a different shaped support structure 25, although this can have the same functionality as support structure 20. Triggering mechanism 48 is in the inactive position and in which a part of housing 14 can be seen. Triggering mechanism 48 can also be seen coupled to drive link 72 and drive arm 74 using connector pins 76.

In use, imaging device 12 is positioned on a patient's skin or tissue at a point of interest, for example where there is suspected malignant growth or other feature which it is desired to image. In this disclosure references may be made to skin only but it is to be understood that the disclosure is equally applicable to tissues and, as a result, any references to skin is to be understood as applying to tissues, and vice versa, where appropriate. In practice, spacer 38 is placed against the skin surface, thereby locating the probe head. In the first instance, triggering mechanism 48 is left untouched, such that marker implement 58 is outside imaging window 50. In some preferred embodiments imaging zone 52 will not be obstructed by guide member 64. Although in some embodiments, a part of the guide member may be in or part of imaging zone 52.

Imaging device 10 can be connected to a processing unit and display such that images obtained from the probe head can be displayed substantially in real time. The physician can move probe head 10 around the skin or tissue in order to determine the perimeter of the skin zone of interest. When the physician identifies, in this embodiment in the centre of imaging zone 52, a perimeter position of interest, the physician can press the triggering mechanism 48, causing marker implement 58 to move from the first, inactive position, through to the second position at the end of the second guide channel section 62. Marker implement 58 moves from the first position to the second position while the apparatus remain in position on the tissue and while imaging window 50 remains in position on the tissue. Marker implement 58 then moves orthogonally along first guide channel section 60 so as to apply a mark at that position. Probe 12 is not moved out of place during this operation and imaging window 50 remains in position on the surface of the skin, such that a mark can be placed precisely at the position of interest. For this purpose, the system can display positioning means, such as crosshairs on the displayed image within imaging zone 52. The crosshairs are indicative of the location within imaging zone 52 where marker implement 58 will apply the mark on the surface of the tissue. This allows the physician to see and target, under image guidance, exactly where in the field of view of the displayed image the mark is being applied. The physician can also move imaging window 50 under image guidance so as to position the crosshairs at the exact location of the clinical feature of interest and apply the mark where the physician intends. Preferably, the crosshairs are displayed in the centre of the displayed image, indicative of the centre point of imaging window 50. A number of other types of positioning means are suitable, including a circular centering point, a centering dot and/or a bullseye.

In other words, a mark can be applied precisely with no, or at least little, parallax error and without having to lift off or move the probe head from its imaging position. In some preferred embodiments, marker implement 58 uses a gentian violet medical grade ink, suitable for surgical marking of skin. However, other suitable medical grade inks can be used.

First guide channel section 60 ensures, or at least increase the likelihood, that marker implement 58 moves orthogonally in the last part of its movement, and therefore along a line normal of the plane of imaging window 50. As a result, a mark can be placed precisely at the centre point even when the skin surface is not completely flat against the front face 42 of spacer 38.

Marker implement 58 is preferably resilient or resiliently held such that pressure applied by marker implement 58 on the skin surface is controlled. In some embodiments, coupling device 64, particularly the part connected to marker implement 58, is resiliently deformable, while in other embodiments marker implement 58 can be held in place be a resiliently deformable coupling device 64 such as a spring or spring-like element (not shown). In other embodiments, marker implement 58 itself can be deformable under pressure, although this is not preferred.

Once a mark has been applied, triggering mechanism 48 can be released, whereupon marker implement 58 is moved back to its inactive position, by resiliency in the assembly. The probe head can be moved again and once another position of interest is identified triggering mechanism 48 is pressed again. In this manner, a plurality of points can be marked on the patient's skin so as to delimit an area for treatment.

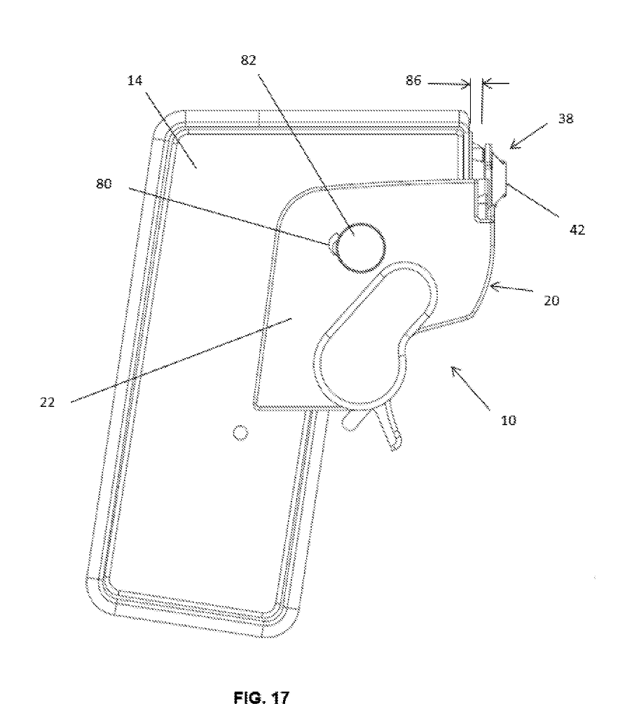

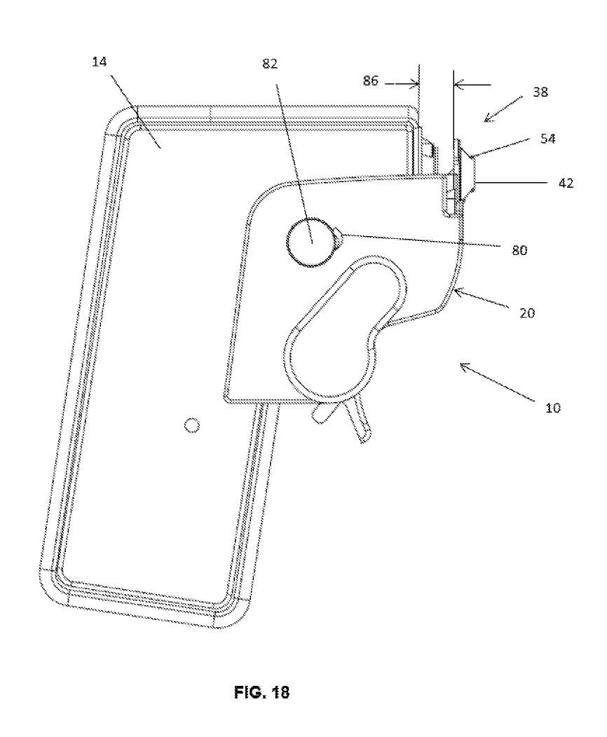

The function and operation of spacer 38 is now described with reference to FIGS. 17 to 22. With reference first to FIGS. 17 and 18, these show marking apparatus support structure 20 in two positions, a back position as in FIG. 17 and an extended position as shown in FIG. 18. The partially toothed slot 80 in housing panel 22 is shown in better detail. When toothed thumbwheel 82 is rotated in the clockwise direction in FIG. 17, the components of marking apparatus 10 are moved away from housing 14 of probe 12, as can be seen in FIG. 18. A comparison of spacer 38 in FIGS. 17 and 18 shows that front face 42 of spacer 38 moves forwardly, away from probe housing 12 between the configurations of FIGS. 17 and 18. The forward movement of support structure 20 causes an increase in the spacing between spacer 38 and probe housing 14, as can be seen by arrows 86.

Thumbwheels 82 can be rotated in the opposite direction to move the components of marking apparatus 10 back and to shorten the spacing between spacer 38 and housing 14.

In practice, the spacer element can be adjustable in length by between 0 millimetres and around 20 millimetres.

For imaging skin cancers such as basal cell carcinoma, spacer element 38 is preferably adjustable in length by around 6 millimetres, in practice by moving the front face 42 of spacer 38 between 5 millimetres from the front face of the probe head to 11 millimetres therefrom. This range of length is particularly suitable when using an OCT imaging device. However, spacer 38 can be adjustable in length by other distances suitable for other imaging technologies. Depending on the imaging probe being used the lengths of the spacer element will need to be adjusted. For some imaging technologies, it is preferable that the spacer remains in the back position as in FIG. 17. Certain imaging technologies will require the spacer to remain in the back position and certain imaging technologies will require spacing between spacer 38 and housing 14. As will be described below, this change in length of the spacer element changes the focal depth of the probe head to image below skin surface. The spacer may be adjustable in length by various distances in order to achieve different focal depth ranges. Spacer 38 can also be adjustable in length by various distances in order to facilitate removal of spacer 38. In practice, the thumbwheels can be turned partially, so as to set the front face 42 of spacer 38 at any depth within the extremes of this range.

It will be appreciated that for imaging other features or symptoms a different depth range may be provided.

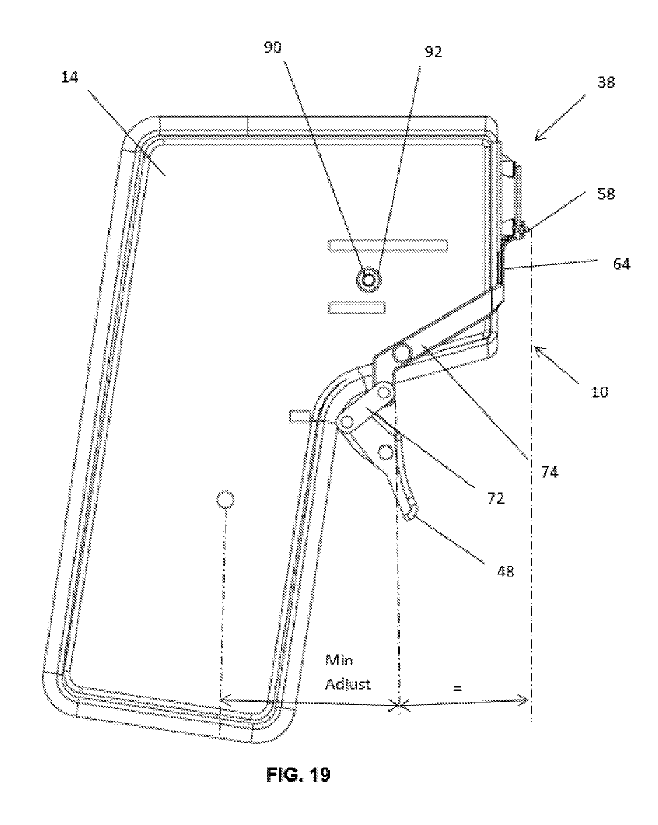

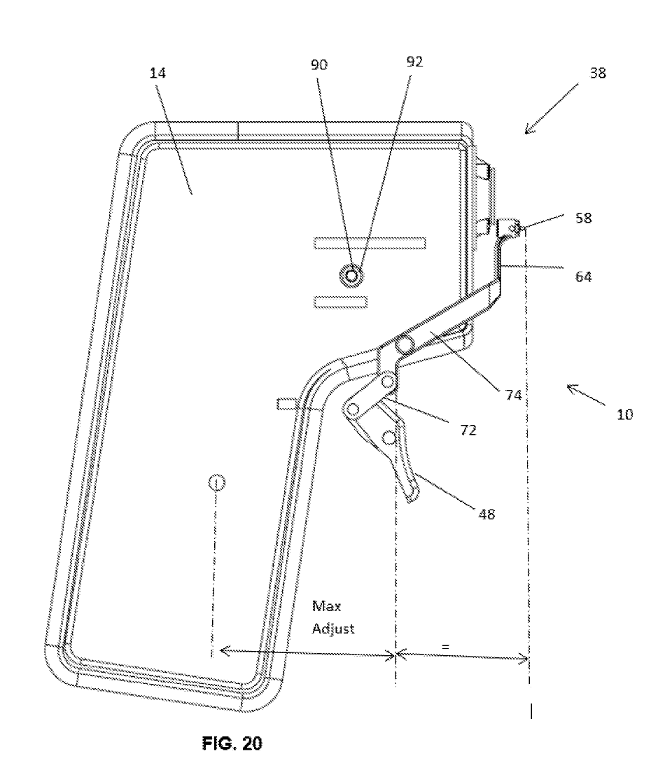

FIGS. 19 and 20 show side elevations of probe housing 14 and of marking apparatus 10 with marker implement 58 with coupling device 64 in the two extreme positions. FIGS. 19 and 20 show the minimum and maximum adjustments of marker implement 58 during spacer 38 adjustment. As can be seen from a comparison of these Figures, the internal components move as one. It is not necessary, therefore, to provide for changes in the relative position of marker implement 58 as support structure 20 is moved position.

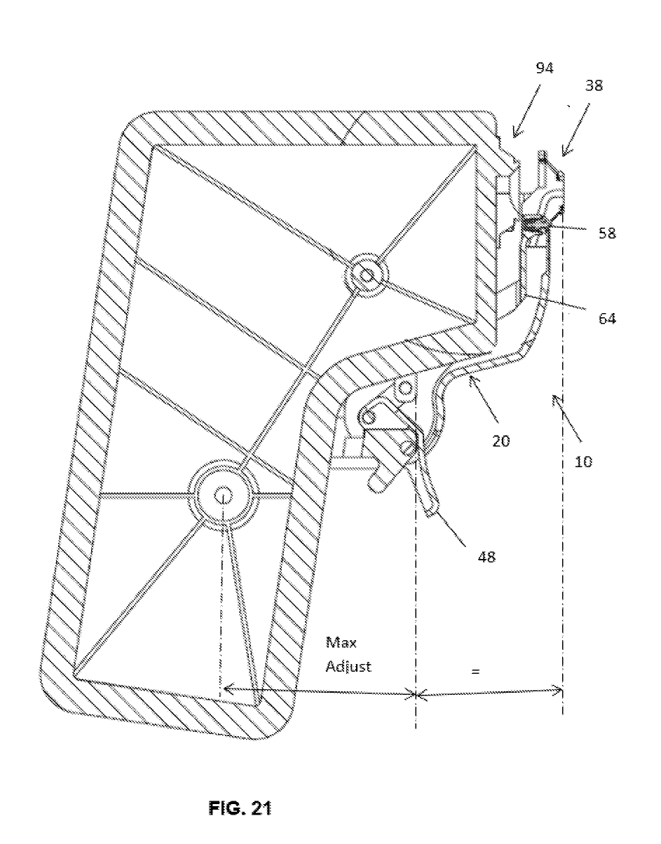

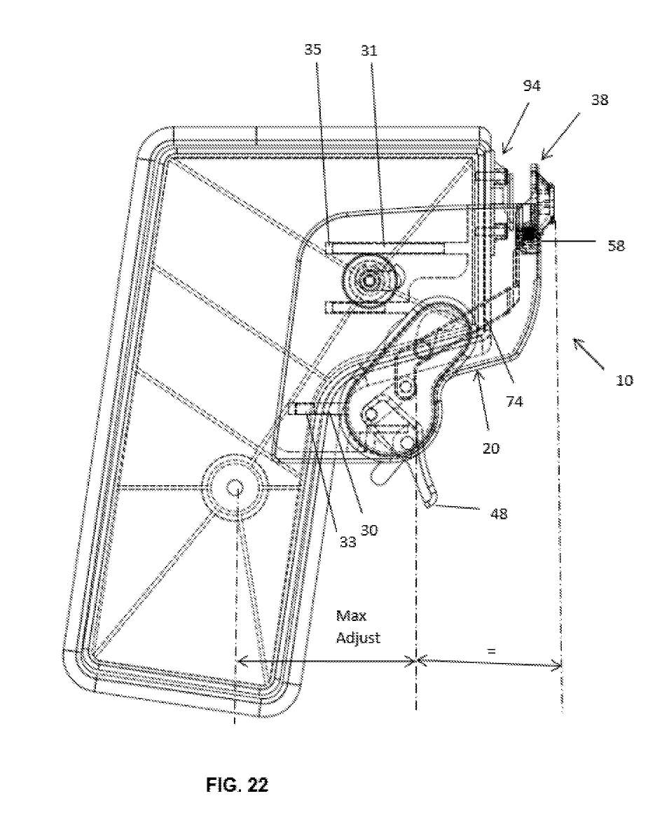

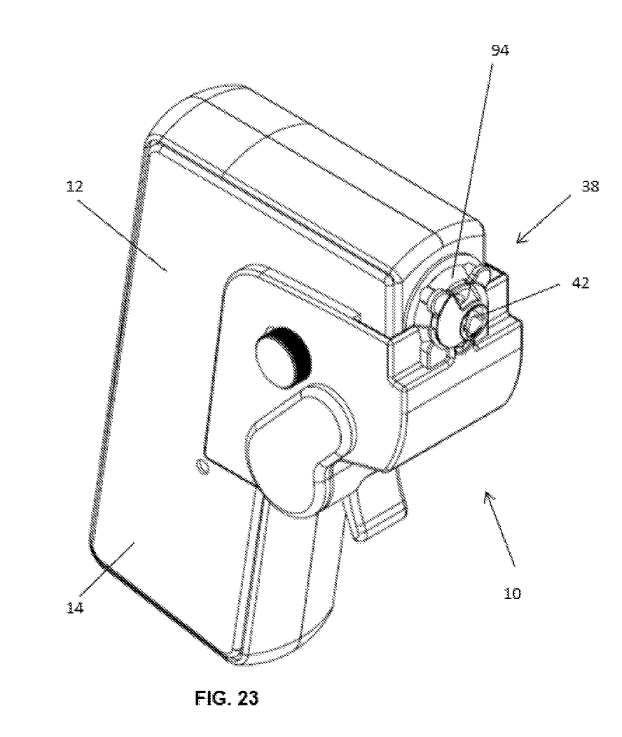

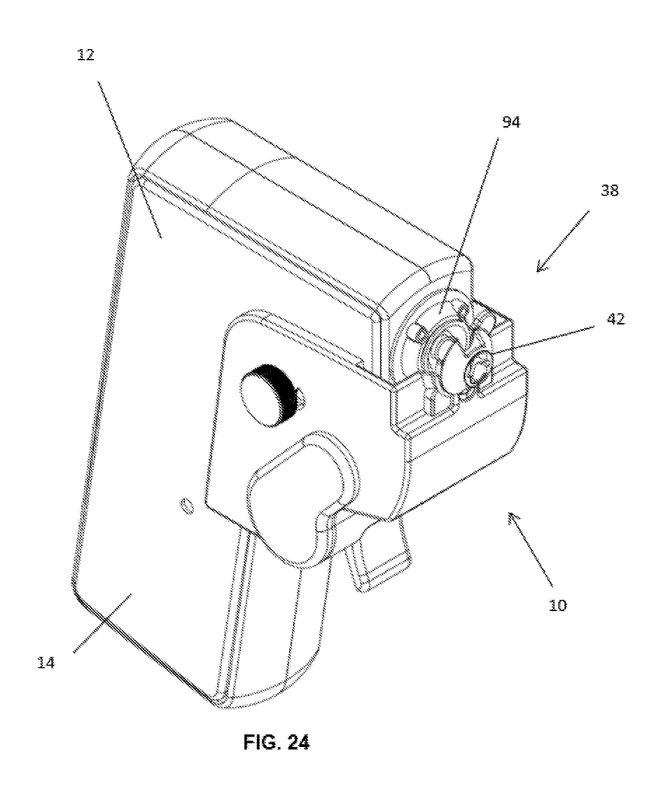

In some embodiments, the left and right thumbwheels 82 are connected together by a spindle (not shown) which can pass through bore 90 in internal tubular support 92 of the two housing halves of probe housing 14. Tubular supports 92 can in practice abut one another, providing rigidity and strength to probe housing 14. FIGS. 21 and 22 are cross sections views in side elevation of device 10 with spacer 38 in the retracted and extended positions and which show clearly how spacer 38 can be elongated so as to change the distance of the front face 42 relative to probe head 94. As can be seen in FIG. 22, spacer 38 moves forward by movement of guide inserts 30 and 31 along guide slots 33 and 35. As drive arm 74 and coupling device 64 are supported on support structure 20 of apparatus 10, when support structure 20 is moved so do the internal components. FIGS. 23 and 24 are front perspective views of device 10 with spacer 38 in the retracted and extended positions.

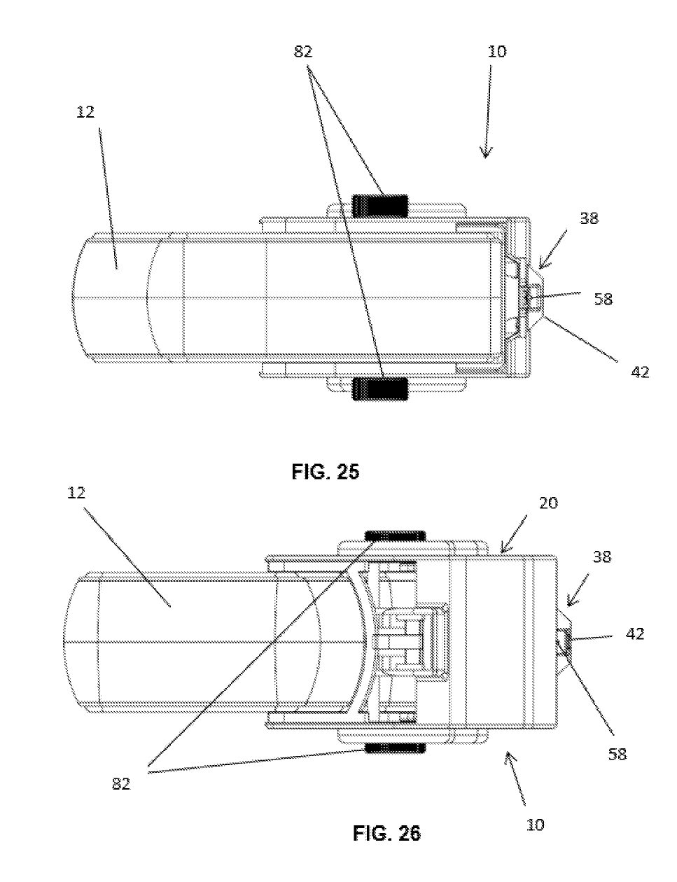

FIGS. 25 and 26 show marking apparatus 10 with spacer 38 in the retracted position. FIG. 25 shows marking apparatus 10 from a top perspective view and FIG. 26 shows marking apparatus 10 from a bottom perspective view. Thumbwheels 82 from moving mechanism 36 position support structure 20 in its most rearward position, closest to imaging probe 12.

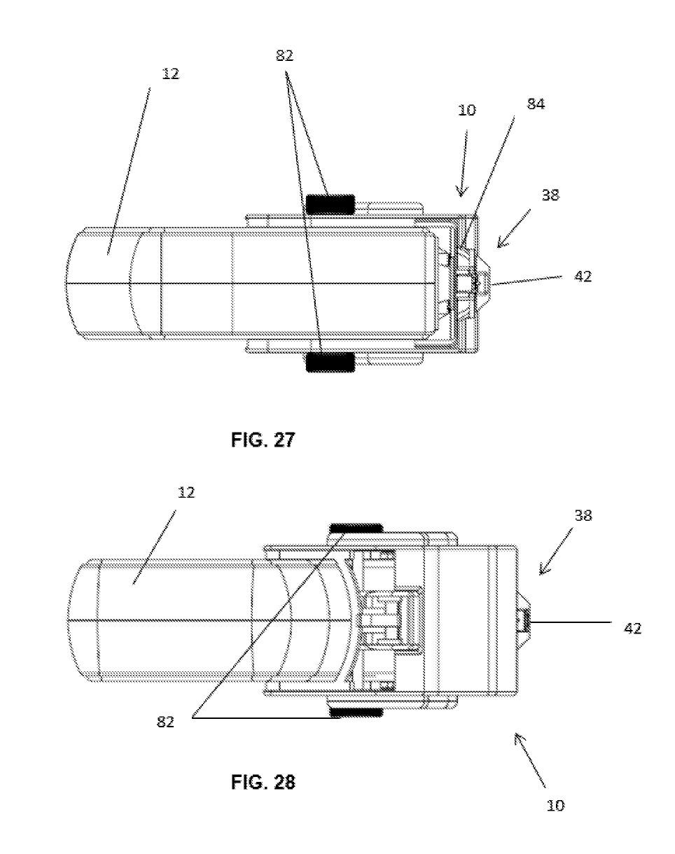

FIGS. 27 and 28 show marking apparatus with spacer 38 in the extended position. FIG. 27 shows a top perspective view of marking apparatus 10 and spacer 38 can be seen in its extended position. FIG. 28 shows a bottom perspective view of marking apparatus 10. In FIGS. 27 and 28, thumbwheels 82 position support structure 20 in its most forward position, with spacer 38 furthest away from imaging probe 12.

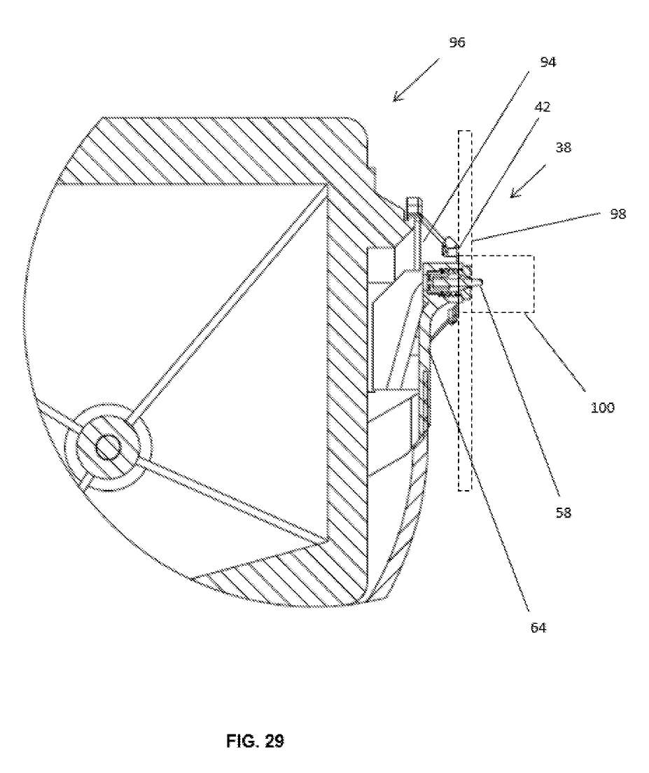

FIG. 29 shows, in schematic view, the effect of changing the spacing between the front face 42 of spacer 38 and probe head 94. Changing this spacing changes the point of focus of probe 96 of imaging device 12. A depiction of a patient's skin surface is shown at 98 in FIG. 29, whereas box 100 represents the depth of the field of view of probe 96, which varies with the distance between the front face 42 of spacer 38 and probe head 94. When the distance is less the point of focus of probe head 94 is further to the right in box 100 as seen in FIG. 29, that is deeper into the skin. On the other hand, when the distance between the front face 42 of spacer 38 and probe head 94 is greater, the point of focus of probe head 94 is further to the left in box 100 as seen in FIG. 29, that is closer or at the skin surface. Thus, as thumbwheels 82 are turned, the focal length of probe 96 can be adjusted to image at different depths. This is useful not only for imaging at different depths within skin or tissue but can also be useful to take into account variations in skin contour, or different imaging techniques.

As explained briefly above, imaging device 12 and apparatus 10 are typically part of an imaging system which includes an imaging processing unit and a display. Such systems are generally known, although without probe 96 and assembly taught herein. Thus, in some embodiments, the disclosed devices and methods can comprise these other elements of an imaging system.

In most embodiments viewing window 50 can simply be an aperture, although it is not excluded that this can be closed by a transparent or translucent panel for example of glass or plastics material. Such a panel can be simply light transmissive although in some embodiments the panel can be at least partially concave or convex to provide a focusing function.

In some preferred embodiments, spacer 38 is integral with the remainder of apparatus 10.

In some embodiments, spacer 38 is a separable component.

In some embodiments, apparatus 10 can be incorporated into probe housing 14, in which case the focal distance of the probe can be changed by adjusting the position of the probe relative to probe housing 14.

In some preferred embodiments, marker implement 58 is a felt-tip nib. In other embodiments, marker implement 58 can be another suitable device, such as inkjet device. It can also be solenoid or hydraulically operated for at least a part of its range of movement.

Marking apparatus 10 can be used also with an ultrasound imaging device, for instance for locating and marking the location and length of a vein.

In some embodiments, marking apparatus 10 can be used with applications where needles need inserting at an accurately predetermined position above a feature of clinical significance, such as when taking a biopsy of the skin or during plastic surgery or during spinal anaesthesia procedures. Device 10 can be used for locating benign lesions or during aesthetic procedures. Apparatus 10 can also be used in dermoscopy and reflectance confocal microscopy, with an opto-acoustic, a reflectance confocal microscopy, a terahertz, a raman spectroscopy imaging device and/or a combination of OCT and reflectance confocal microscopy imaging device, as well as in any other medical procedures for which imaging and marking of an imaged body part is appropriate. Spacer 38 can be adaptable depending upon the imaging technology being used, in particular whether spacer 38 is movable between a back position and an extended position and the distance the front face 42 of spacer 38 can be moved from the front face of the probe head.

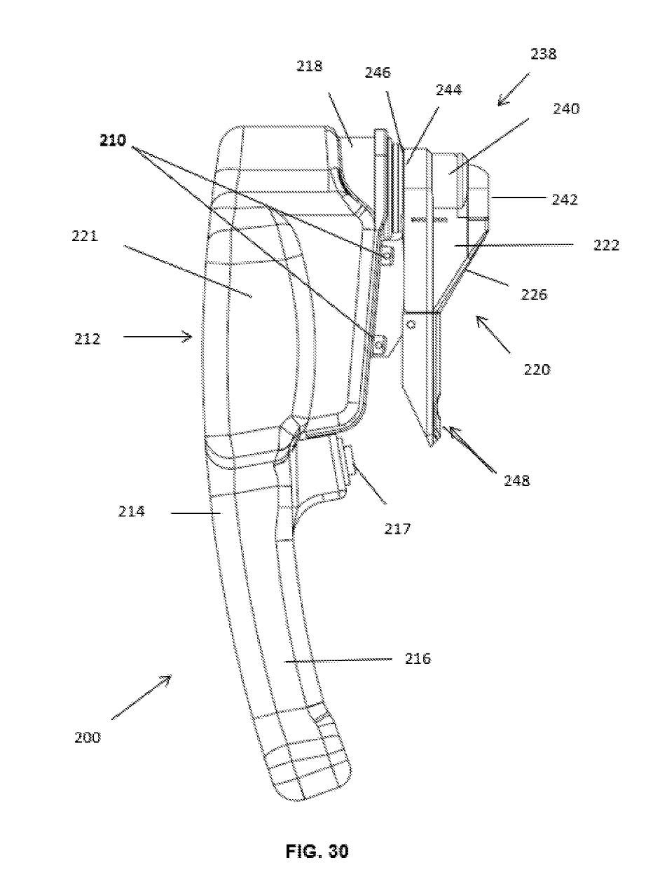

The features described above can be implemented in a variety of ways. Another embodiment of marking an apparatus is described below. It is to be understood that features of the above embodiments can be used with the embodiment described below and vice-versa. The following embodiment, shown in FIGS. 30 to 44 is particularly suitable for use with an RCM imaging system.

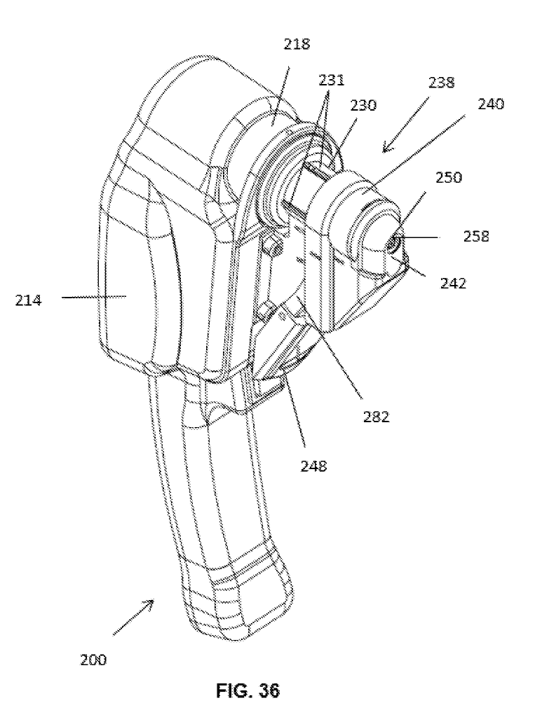

FIG. 30 is a view in side elevation of another embodiment of marking apparatus 200, including an imaging device or probe 212 for imaging tissue. Imaging probe 212 can include probe housing 214 which is differently shaped from housing 14 of the previous embodiments, although it has a similar functionality as housing 14. As with the embodiments described above, probe housing 214 can include side walls 221 and 223, head 218 and hand grip 216 which in this embodiment is curved to facilitate gripping by a user. Head 218 can include a front face 246. Marking apparatus 200 can include spacer 238, provided at the front of apparatus 200 and designed to fit and attach to imaging probe 212, specifically at head end 218 of probe housing 214. In at least some embodiments, spacer 238 can move forwardly from probe 212 as shown in FIG. 36 and as described further below. Spacer 238 can include housing 240 which includes a front face 242, a back face 244 and two side walls 222 and 224 joined by front wall 226. Marking apparatus 200 can also include a triggering mechanism or trigger 248 positioned at the front of apparatus 200. In some embodiments, push button 217 is also provided positioned at the top of hand grip 216 on the front of housing 214. In at least some embodiments, trigger 248 and button 217 are positioned such that a user gripping marking apparatus 200 with one hand can easily reach and actuate both trigger 248 and button 217 with a finger. Connecting pins 210 can also be included on the front of housing 214, either side of spacer 238, for attaching marking apparatus 200 to imaging probe 212. This is often required in embodiments where marking apparatus 200 is retrofitted to imaging probe 212. In other embodiments, marking apparatus 200 can be integral with imaging probe 212, in which case connecting pins 210 may not be required.

FIG. 31 is a view in front elevation of the apparatus shown in FIG. 30. Spacer 238 includes window 250 on front face 242. As with previous embodiments, window 250 provides imaging zone 252 within its perimeter. Trigger 248 includes oval indentation 249. A user gripping marking apparatus 200 can position a finger on indentation 249 to aid gripping of trigger 248.

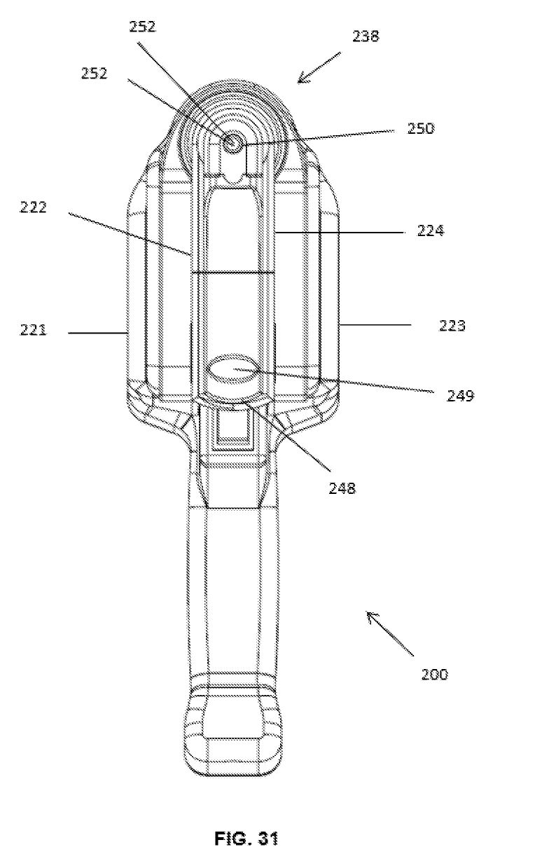

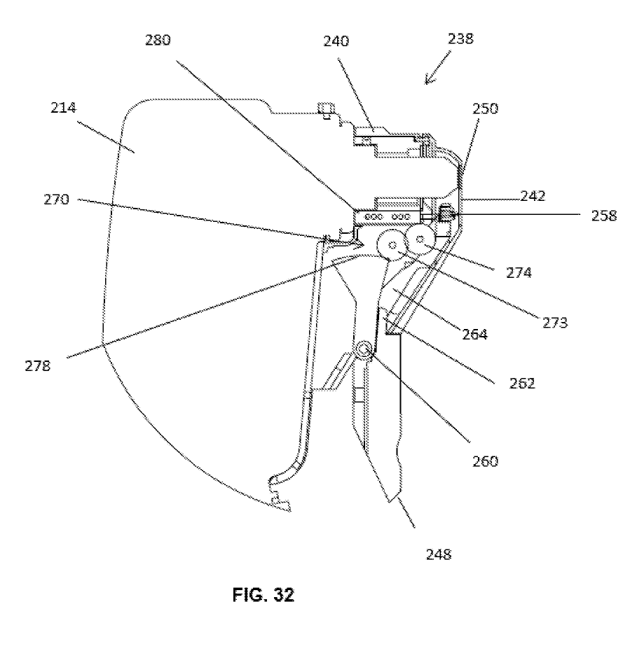

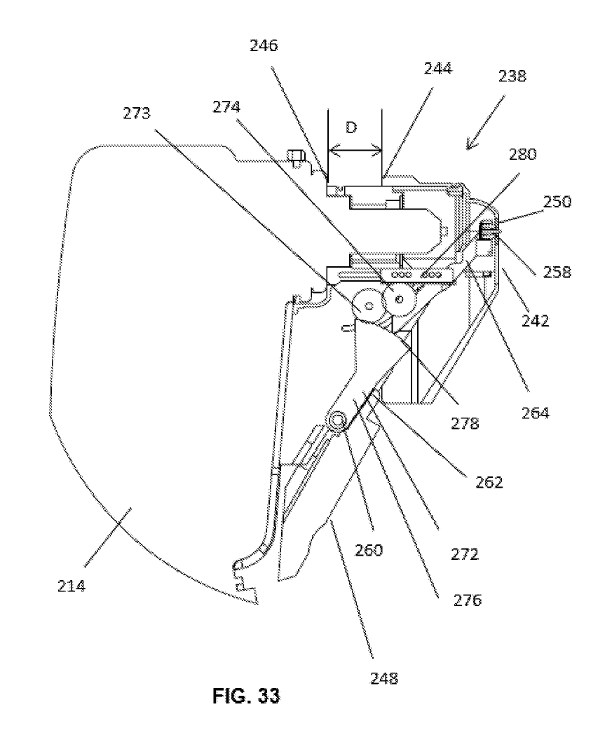

FIGS. 32 and 33 show part cross-sectional views of marking apparatus 200, including marker implement 258 which can be of the form described in previous embodiments. FIG. 32 shows marker implement 258 in a first position, outside imaging window 250, whereas FIG. 33 shows marker implement 258 extending through imaging window 250 and in a position in which it can apply a mark to skin abutting the front face 242 of spacer 238.

FIG. 32 shows trigger 248 in the inactive position. Trigger 248 can be seen coupled to gear system 270 including drive link 272 and two gears 273 and 274. Drive link 272 can include arm 276 and curved head 278 and can be pivotable about pin 260. Curved head 278 can include teeth or cogs which mesh with teeth on gear 273. The teeth or cogs are not visible in the Figures. The teeth on gear 273 in turn mesh with teeth on gear 274, while the teeth on gear 274 mesh with teeth on push link 280. In some embodiments, push link 280 is coupled to the housing 240 of spacer 238 such that push link 280 and housing 240 move together.

A coupling device or link 264 has one end connected to marker implement 258 and the other end connected to trigger 248. Coupling link 264 connects to trigger 248 via connecting pin 262 (not shown). Coupling link 264 pivots about pin 262.

Marking implement 248 is in the first position outside imaging window 250 and retracted from the front face 242 of the spacer. In this first position or the non-use position, marker implement 258 sits within housing 240 of spacer 238.

FIG. 33 shows trigger 248 in the active position, in which trigger 248 has been pressed towards the body of probe housing 214. This action causes drive link 272 to pivot about pin 260 and move forwardly, as shown in the FIG. 33. The teeth on curved head 278 cause turning of gear 273 in an anti-clockwise direction, which in turn cause turning of gear 274 in a clockwise direction. Turning of gear 274 in a clockwise direction causes push link 280 to move forwardly, moving housing 240 forwardly and extending spacer 238 forwardly from probe housing 214. By the forward movement of housing 240, this causes an increase in spacing between the back face 244 of spacer 238 and the front face 246 of probe head housing 218, as can be seen by the arrow indicated by ‘D’. Movement of trigger 248 into the active position also allows coupling link 264 to pivot about pin 262, moving marker implement 258 forwardly and upwardly into the extended (or in-use) position and in imaging zone 252 of imaging window 250. When in the extended position, marker implement 258 extends through imaging window 250 to apply a mark to the surface of skin abutting the front face 242 of spacer 238.

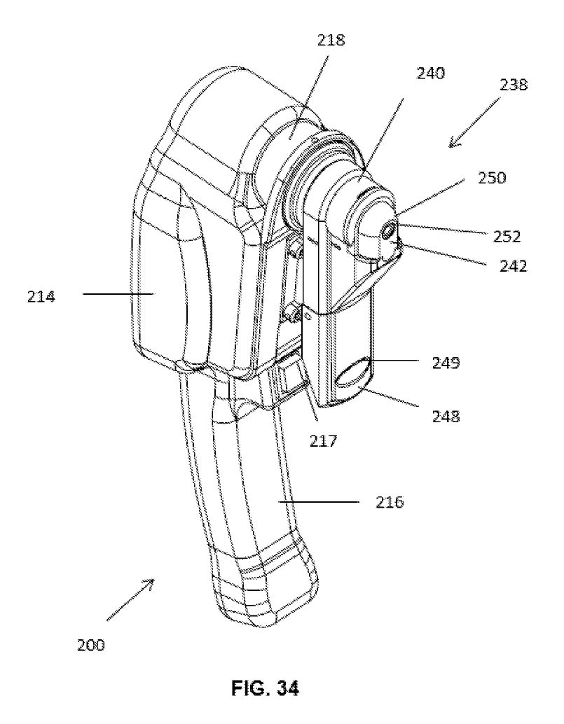

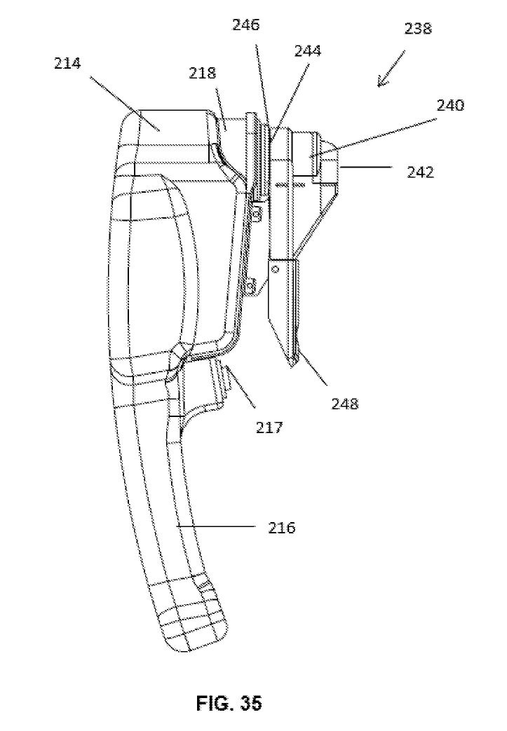

FIGS. 34 and 35 show different views of marking apparatus 200 with trigger 248 in the inactive position. Marker implement 258 is outside imaging window 250 and within housing 240. Spacer 238 is in the retracted position.

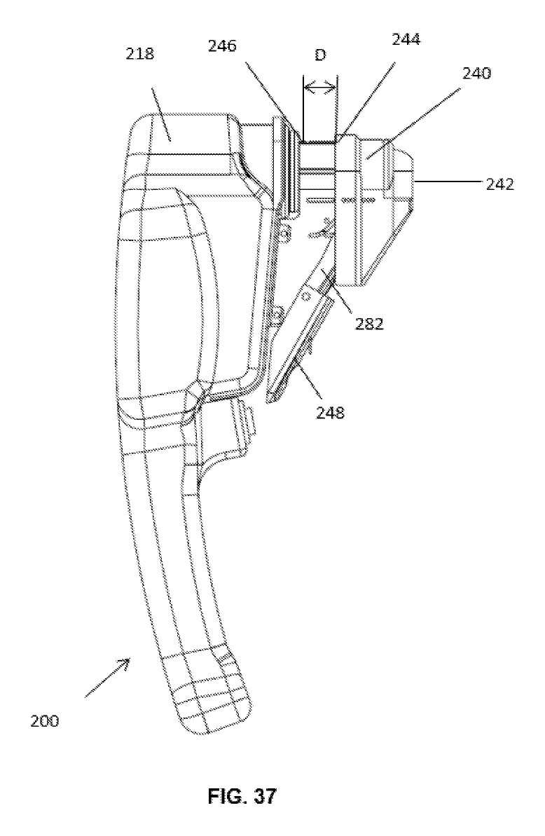

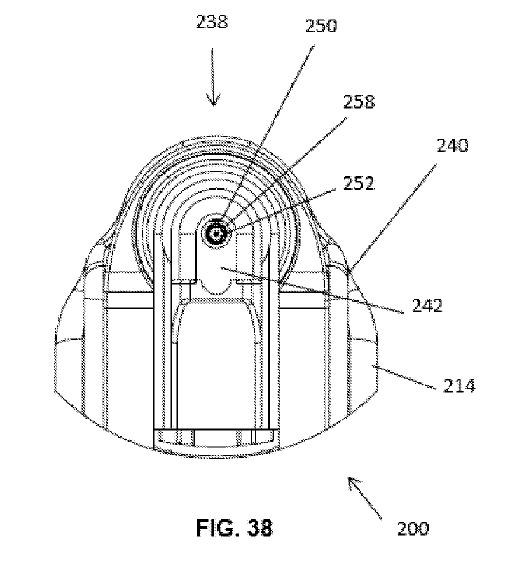

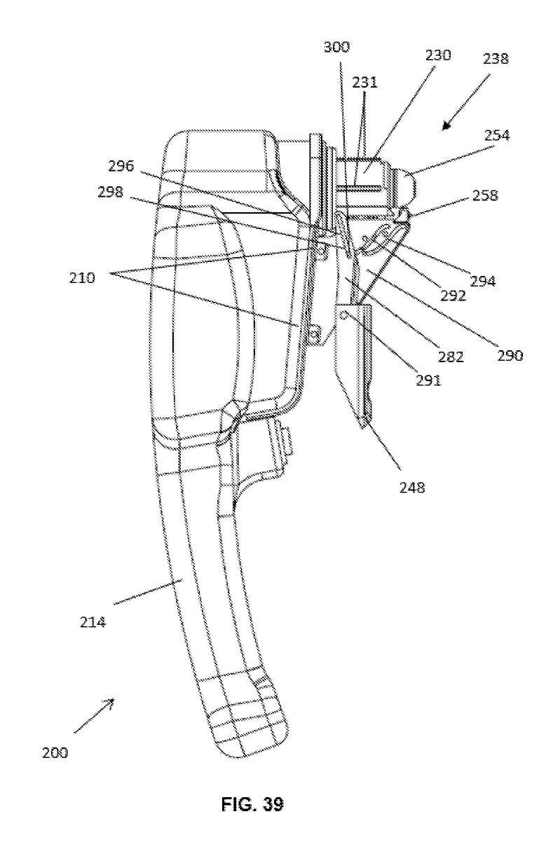

FIGS. 36 to 38 show apparatus 200 with marker implement 258 in the active or extended position. Trigger 258 has been pressed towards the body of housing 214 into the active position. Gear system 270 is not shown and trigger 248 can be seen coupled to drive arm 282 (described further below). As can be seen in FIGS. 36 and 37, spacer 238 is in the extended position. Housing 240 of spacer 238 is telescopically extendable and retractable. Housing 240 can include collar 230 which extends forwardly from head 218. Collar 230 can include guides 231 which are in the form of elongate ribs which keep spacer 238 aligned with marker implement 200 as it telescopes out from probe body 214. FIG. 38 shows marker implement 258 positioned in the centre of imaging zone 252, as it is in the extended, in-use, position.

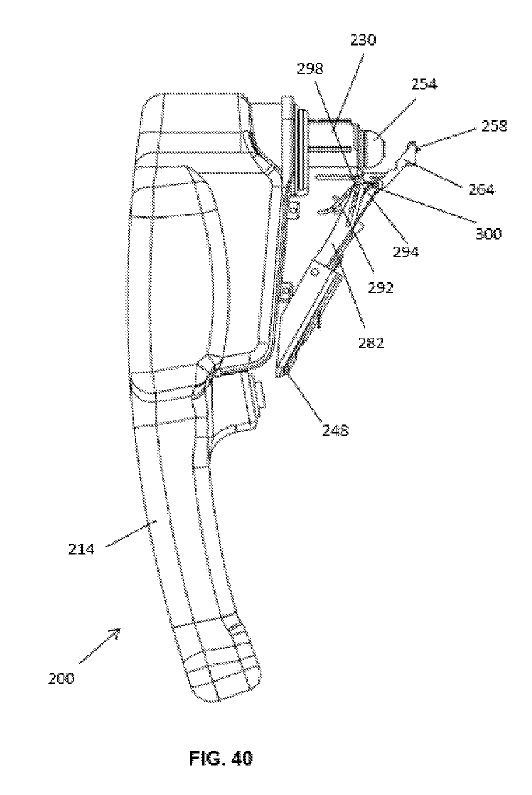

FIGS. 39 and 40 show side elevations of marking apparatus 200 with marker implement 258 in the two extreme positions. Housing 240 has been removed and gear system 270 is not shown so that the internal components of spacer 238 can be seen. The probe includes domed lens cover 254 located on the front of probe head 218. Spacer 238 includes internal guide structure 290 which includes guides 292 and 294 in this embodiment in the form of S-shaped slots within internal support wall 290. In this embodiment the two guides 292 and 294 are the same shape and are equidistant over at least a part of their extent. Drive arm 282 is fixedly connected to trigger 248, optionally via pin 291 or otherwise fixed thereto. Both trigger 248 and drive arm 242 can pivot about pin 291. Drive arm 282 includes guide slot 296 and guide pin 298 connects guide slot 296 to guide slot 292. In this position, pin 298 sits in the lower bend of slot 292. A second guide pin 300 connects guide slot 294 to coupling link 264 to which marker implement 258 is coupled. Similarly, pin 300 sits in the lower bend of slot 294. In some embodiments, the internal components of spacer 238 are two sided, i.e. a second drive arm can be provided on the other side of internal guide structure 290 with equivalent components provided in a mirror image arrangement.

FIG. 40 shows marker implement 258 in the extended position. Trigger 248 is pressed towards body 214 causing drive arm 282 to pivot about pin 291 and move into a forward position, as shown. Drive arm 282 moves pin 298 into the upper bend of slot 292. The action cause movement of pin 300 in slot 294, moving pin 300 into the upper bend of slot 294. Movement of pin 300 causes coupling link 264 to move forwardly and upwardly, moving marker element 258 beyond domed lens cover 254 and causing it to extend into imaging zone 252.

Marking apparatus 200 is used in a similar way to marking apparatus 100. Spacer 238 is placed against the skin surface and probe head 218 is moved around the skin in order to determine a skin zone of interest. A user or physician can press push button 217 to capture a screenshot of the image viewed by the probe. The screenshot is obtained without the physician having to move or lift the imaging window off the skin surface and while the apparatus remains in position on the tissue. The screenshot can therefore be accurately captured at the exact location of any identified features of interest. As a perimeter position of interest is identified, the physician can press trigger 248, causing simultaneous movement of marker implement 258 into the extended (in-use) position, as well as of spacer 238 into the extended position. Movement of trigger 248 from the inactive position to the active position causes drive link 272 to pivot forwardly, turning gear 273 in an anti-clockwise direction and gear 274 in a clockwise direction and moving push link 280 forward. The forward movement of push link 280 moves spacer 238 forwardly from marker body 214. The forward movement of spacer 238 causes the front face 242 to push the surface of the skin away from probe 212 and results in a change in spacing between probe head 218 and face 244 and thus of probe head 218 and front face 242 with imaging window 250. This can be necessary for certain types of imaging techniques, including for example reflectance confocal microscopy (RCM), Optical coherence tomography (OCT) and/or combinations of OCT and RCM imaging techniques amongst others.

In at least some embodiments, movement of trigger 248 into the active position at the same time causes drive arm 282 to pivot about pin 291, driving pin 298 forwardly and upwardly in slot 292. This causes pin 300 to move forwardly and upwardly in slot 294, moving coupling link 264 and moving maker implement 258 into imaging zone 252 and extending it beyond imaging window 250 so as to apply a mark to the surface of the skin. Marker implement 258 moves from the first position to the extended position while marking apparatus 200 remains in position on the tissue and while imaging window 250 remains in position on the tissue at the location of the identified feature of interest. Marker implement 258 can therefore apply a mark to the surface of the skin, marking the point of interest, without having to move probe head 218 or imaging window 250 and without having to lift imaging window 250 off the surface of the skin thereby accurately indicating the location of the identified feature of interest. Marker implement 258 can therefore apply the mark to the skin while probe 212 and marking apparatus 200 remain aligned as the physician had intended and with imaging window 250 aligned and remaining in contact with the tissue surface. This allows the physician to apply the mark under image-guidance. This also allows the physician to image the feature of interest at the same time as the mark is being applied.

As with previous embodiments, once a mark has been applied, trigger 248 can be released, whereupon marker implement 258 is moved back to its first non-use position by resiliency in the assembly. As marker implement 258 returns to the non-use position, spacer 238 moves back to its original retracted position.

As with previous embodiments, the system can display positioning means, such as crosshairs on the displayed image within imaging zone 252. The crosshairs are indicative of the location within imaging zone 252 where marker implement 258 will apply the mark on the surface of the tissue. This allows the physician to see and target, under image guidance, exactly where in the field of view of the displayed image the mark is being applied. The physician can also move imaging window 250 under image guidance so as to position the crosshairs at the exact location of the clinical feature of interest and apply the mark where the physician intends.

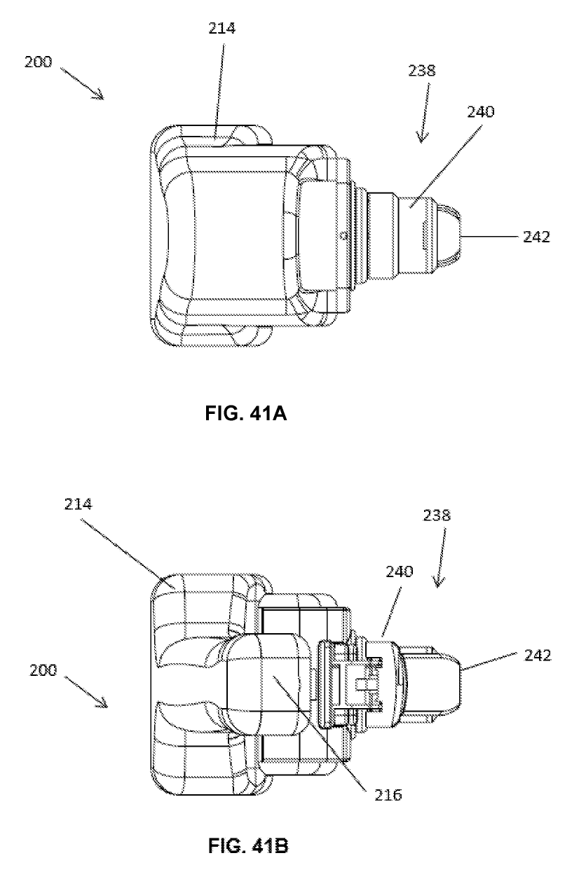

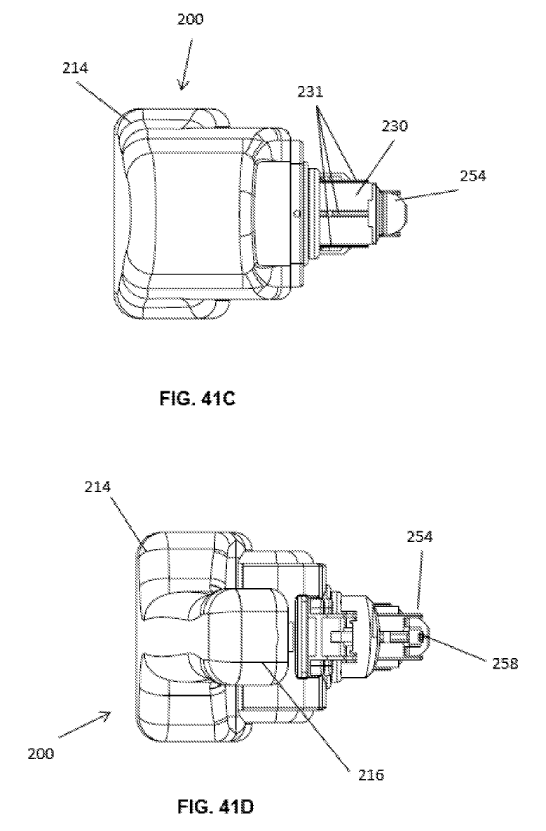

FIGS. 41A to 41D show further views of marking apparatus 200 with marking implement 258 in the first position. Spacer 238 is in the retracted position. FIG. 41A and FIG. 41B show marking apparatus 200 from a top and bottom view respectively. FIG. 41C and FIG. 41D show marking apparatus 200 from a top and bottom view respectively, with part of spacer housing 240 removed.

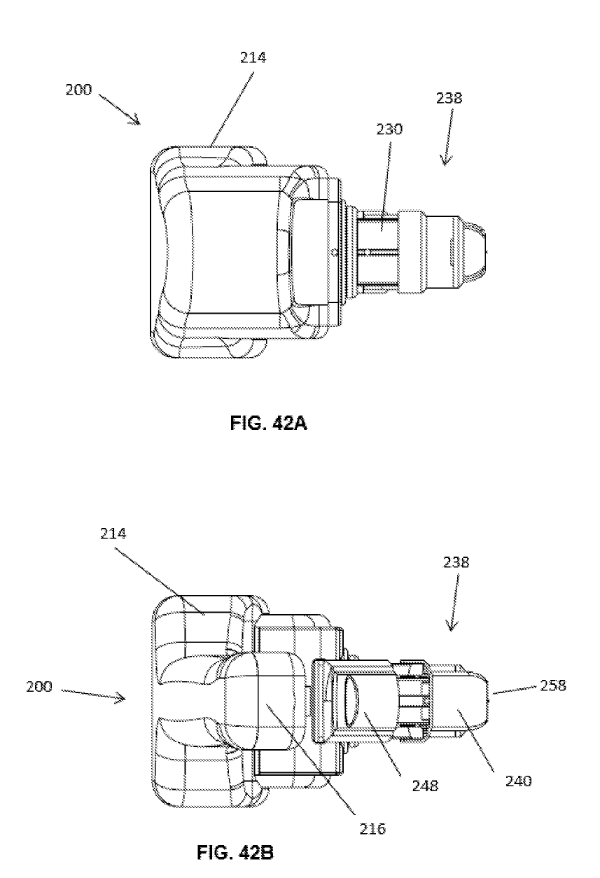

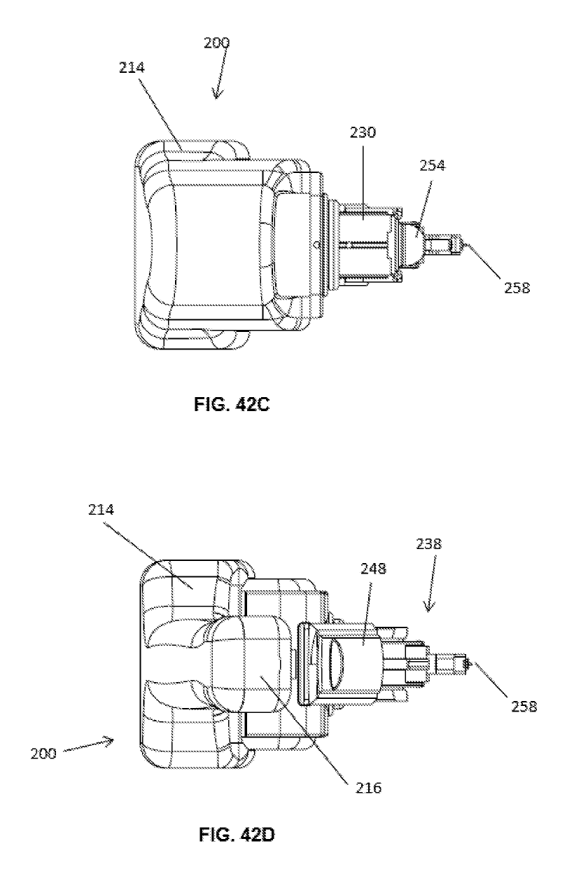

FIGS. 42A to 42D show further views of marking apparatus 200 with marker implement 258 in the extended position. Spacer 238 is in the extended position. FIG. 42A and FIG. 42B show marking apparatus 200 in a top and bottom view respectively. FIG. 42C and FIG. 42D show marking apparatus 200 from a top and bottom view respectively, with part of spacer housing 240 removed. As can be seen most clearly in FIGS. 42B and 42D, trigger 248 is pressed towards the body of probe 214.

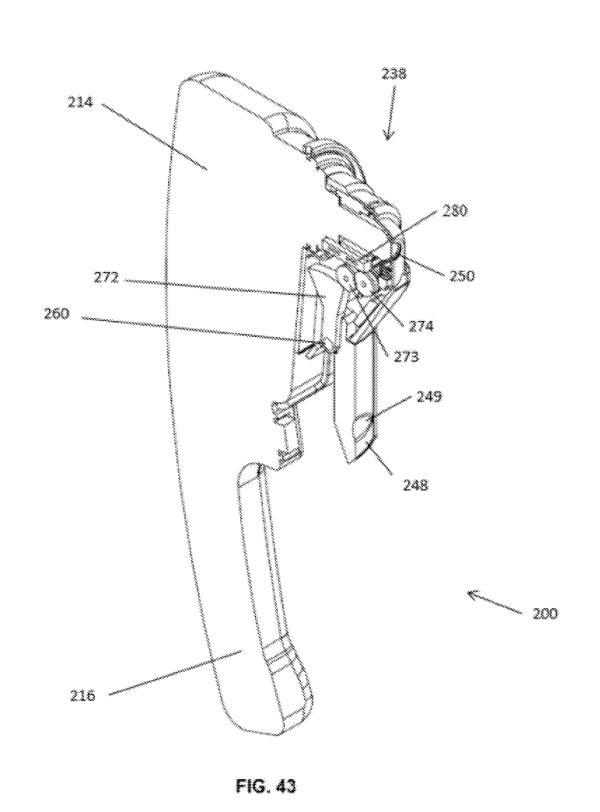

FIG. 43 shows a cross sectional front perspective view of marking apparatus 200.

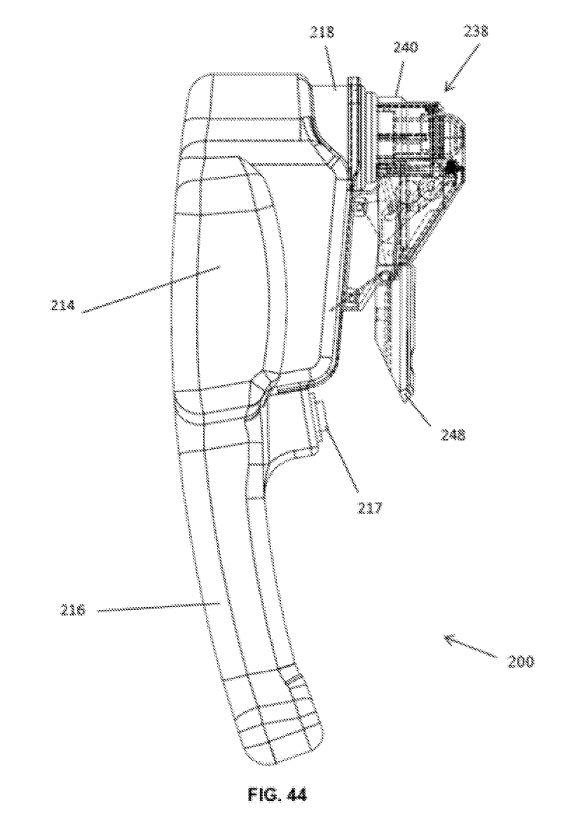

FIG. 44 shows a side elevation of marking apparatus 200 with the internal components of spacer 238 shown in dotted lines.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

What is claimed is:

1. A marking apparatus for marking a tissue imaged through an imaging device comprising:
(a) a viewing element comprising an imaging window providing an imaging zone through which said tissue can be imaged;
(b) a marker implement; and
(c) a coupling device connected to said marker implement;
wherein said coupling device is configurable to position said marker implement in a first position substantially out of said imaging window and a second position in said imaging zone of said imaging window,
wherein said marker implement can apply a mark when in said second position to said tissue in said imaging zone wherein said marker implement moves from said first position to said second position while said marking apparatus remains in position on said tissue.

2. The marking apparatus of claim 1 further comprising:
(d) a guide member to which said marker implement is coupled, wherein said guide member is structured to move said marker implement in a direction substantially orthogonal to said imaging window.

3. The marking apparatus of claim 2, wherein said guide member is operable to move said marker implement so as to extend through said imaging window to apply a mark to said tissue.

4. The marking apparatus of claim 2, wherein said guide member comprises a first guide channel section substantially orthogonal to said imaging window.

5. The marking apparatus of claim 2, wherein said guide member comprises a second guide channel section substantially parallel to said imaging window, said second guide channel section providing for movement of said marker implement in a direction substantially parallel to said imaging window from said first position to said second position.

6. The marking apparatus of claim 1, wherein said marking apparatus is integral with an imaging device.

7. The marking apparatus of claim 1, wherein at least said viewing element and said imaging window are integral with a casing of said imaging device.

8. The marking apparatus of claim 1, wherein said marking apparatus is attachable to an imaging device.

9. The marking apparatus of claim 8, wherein said marking apparatus includes a support structure attachable to a casing of an imaging device.

10. The marking apparatus of claim 1, wherein said marker implement is removable from said marking apparatus.

11. The marking apparatus of claim 1, wherein said viewing element is a spacer element comprising:
(i) a front face which incorporates said imaging window.

12. The marking apparatus of claim 11, wherein said spacer element further comprising:
(ii) a rear face; and
(iii) a length adjusting mechanism which can adjust a distance between said front face and said rear face; wherein a change in said distance effects a change in focal position of said imaging device.

13. The marking apparatus of claim 11, wherein said spacer is configured to extend forwardly from said imaging device into an extended position.

14. The marking apparatus of claim 13, further comprising:
(d) a gear system to which said spacer is coupled, said gear system being operable to move said spacer forwardly into said extended position.

15. The marking apparatus of claim 1 further comprising:
(d) a push button coupleable to said imaging device and configured to capture a screenshot of the image viewed by said imaging device while said marking apparatus remains in position on said tissue.

16. The marking apparatus of claim 1, including positioning means displayed within the imaging zone, wherein said positioning means are indicative of the location where the marker implement is able to apply a mark to tissue.

17. A system for imaging a tissue and marking an imaged tissue said system comprising:
(a) an imaging device;
(b) a marking apparatus coupled to said imaging device comprising;
(i) a viewing element including an imaging window providing an imaging zone through which said tissue can be imaged;
(ii) a marker implement;
(iii) a coupling device to which said marker implement is connected; said coupling device configured to position said marker implement in a first position substantially out of said imaging window and a second position in said imaging zone of said imaging window;
(c) a display system coupled to said imaging device and through which images obtained from said imaging device can be displayed;
wherein said marker implement can apply a mark when in said second position to said tissue in said imaging zone when said marker implement moves from said first position to said second position while said apparatus remains in position on said tissue.

18. A method of using said marking apparatus according to claim 1 in cancer treatment, plastic surgery, benign lesions, aesthetic procedures or biopsy procedures.

19. A method of using said system according to claim 17 in cancer treatment, plastic surgery, benign lesions, aesthetic procedures or biopsy procedures.

20. A configurable viewing element for an imaging device comprising:
(a) a spacer element comprising:
(i) a front face with an imaging window disposed in said front face providing an imaging zone through which a tissue can be imaged; and
(ii) a rear face,
(b) a length adjustment mechanism coupled to said spacer element and operable to adjust a distance between said front face and said rear face to effect a change in a focal position of said imaging device.
